(19) United States
(12) Patent Application Publication
(10) Pub. No.: US 2025/0302416 A1
(43) Pub. Date: Oct. 2, 2025

(54) SYSTEMS AND METHODS FOR GENERATING AND PRESENTING PREDICTIVE SYNTHETIC X-RAY IMAGES

(71) Applicant: Stephen B. Murphy, Winchester, MA (US)

(72) Inventor: Stephen B. Murphy, Winchester, MA (US)

(21) Appl. No.: 19/093,563

(22) Filed: Mar. 28, 2025

Related U.S. Application Data
(60) Provisional application No. 63/572,010, filed on Mar. 29, 2024.

(51) Int. Cl. A61B 6/00; A61B 6/03; A61B 6/46; A61B 6/50; G06T 17/00; G06T 19/20; G16H 10/60; G16H 20/40

(52) U.S. Cl. CPC A61B 6/5235; A61B 6/032; A61B 6/462; A61B 6/505; A61B 6/5205; G06T 17/00; G06T 19/20; G16H 10/60; G16H 20/40; A61B 2505/05; G06T 2210/41; G06T 2219/2004; G06T 2219/2016

(57) ABSTRACT

A computer-implemented method is disclosed for generating and displaying predictive synthetic digital radiographs (DRs). The method comprises receiving pre-operative volume data for at least a portion of a patient's anatomy, the data including voxels with radiographic density or other volumetric values. A 3D model of the anatomy is generated, and a 3D model of an implant is placed at a planned position or orientation within the anatomical model. The post-implantation model is translated into planned post-implantation volume data by changing the voxel values corresponding to the implant. One or more predicted synthetic DRs are created from this volume data and presented on a display to show the implant in its planned position and/or orientation.

### CROSS-REFERENCE TO RELATED APPLICATIONS

This application claims priority to and the benefit of U.S. Provisional Application Ser. No. 63/572,010, filed Mar. 29, 2024, the content of which is incorporate herein by reference in its entirety.

### BACKGROUND

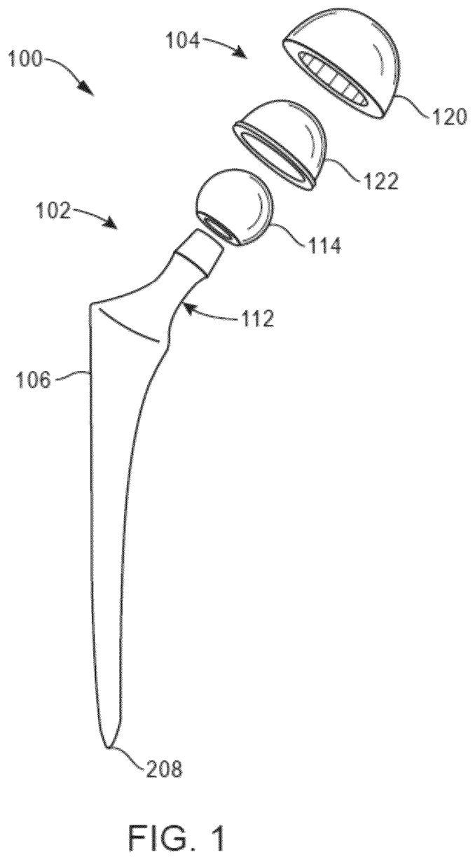

Human hip joints can suffer deterioration, for example, due to aging, deformity, illness, or injury. In total hip replacement or total hip arthroplasty (THA), orthopedic prosthetic implants are used to replace some or all of a hip joint in order to restore its use. FIG. 1 is an exploded view of an example hip implant 100. The hip implant 100 may include a femoral assembly 102 and an acetabular assembly 104. The femoral assembly 102 may include a femoral hip stem 106, a neck portion 112, and a femoral head 114. The acetabular assembly 104 may include an acetabular cup 120 and a liner 122 that fits within the acetabular cup 120. During the surgery, a portion of the patient's native femur including the femoral head and a portion of the femoral neck is resected and replaced with the femoral assembly. A portion of the femoral hip stem is positioned within a femoral canal of the patient's femur. The cup component and liner are implanted in the patient's acetabulum and the femoral head is received within the cup component.

Exemplary hip implants include the Synergy hip system from Smith & Nephew, Inc. of Memphis, Tennessee, the Summit hip system available from Depuy Orthopaedic, Inc. of Warsaw, Indiana, the Epoc Hip System available from Biomet, Inc. of Warsaw, Indiana, the G7 OsseoTi acetabular components and the Avenir Complete femoral components from Zimmer Biomet Holdings, Inc. of Warsaw, IN, among others.

When a hip joint is replaced, changes in leg length, offset, and/or anterior-posterior (AP) position may occur. Leg length refers to the longitudinal extent of the leg, and may be measured, e.g., from a location on the pelvis down to some location along the leg, such as a point on the femur. Offset refers to the lateral or transverse dimension through the hip. The AP position refers to changes along an axis orthogonal to the longitudinal and lateral or transverse axes. Large changes, e.g., 10 mm or more, in leg length, offset, and/or AP position as a result of hip replacement surgery can be desirable or undesirable. For example, if a patient's legs are of equal length before surgery, and the leg of the hip being operated on is lengthened, such that it is 10 mm longer, such an outcome may be undesirable as it can result in tightness and discomfort for the patient. On the other hand, shortening the overall leg length during hip replacement can lead to an unstable hip joint as a result of looser soft tissues, potentially leading to repeated hip dislocations and the need for revision, i.e., corrective surgery.

Accordingly, the position and orientation of the prosthetic hip components is of critical importance in achieving successful THA. The cup component, which has a partially spherical form, may have a center and the position of the cup component may be defined as the x,y,z coordinates of the cup center. The orientation of the cup component may be defined relative to a cup axis, e.g., a line passing through the cup center and perpendicular to the plane of the cup's opening face. Acetabular cup position is traditionally described in terms of the position of the center of rotation, anteversion, and inclination (also referred to as abduction). Improper acetabular cup placement is associated with higher dislocation rates, range of motion (ROM) limitations due to impingement, edge-loading, uneven component wear and, ultimately, higher rates of revision.

Prior to surgery, a surgeon may choose particular hip components, and may plan their position within the hip in order to accomplish a particular goal for the surgery, such as optimizing the changes in leg length, offset, and/or AP position for the patient. In some cases, optimizing the changes may mean minimizing changes to leg length, offset, and/or AP position resulting from the surgery. In other cases, it may mean achieving particular changes to leg length, offset, and/or AP position. The particular components and their locations and orientations may be selected based on patient-specific data, such as patient anatomy.

THA is typically performed using either a posterior, superior, lateral, anterolateral or anterior approach to the hip joint. For the anterior approach, the patient is placed in a supine position. The surgeon may use intraoperative fluoroscopy to assist in placing the prosthetic components at the planned positions. Fluoroscopy is a type of medical imaging that displays digital radiographs instantly on a display to the surgeon during the procedure. The radiographs may be individual images or a stream of images. Typically, a portable fluoroscopy machine having an x-ray source and a detector mounted on a C-shaped arm is used. The C-arm allows the x-ray source (and detector) to be rotated about two axes to obtain a desired image. During THA, a hip stem may be placed in the femur and a cup component and liner may be implanted into the acetabulum. The femoral head may then be inserted into the acetabular cup in a trial reduction of the hip. The hip stem may be a trial hip stem while the cup component may be the final cup component. Nonetheless, in some cases, a final hip stem and/or a trial cup component may be utilized in the procedure. PosteroAnterior (PA) or Anteroposterior (AP) fluoroscopic images of the hip may be taken and displayed to assist the surgeon in checking the position and/or orientation of the components, e.g., whether the cup component meets the desired anteversion and inclination values. In the PA or AP fluoroscopic images, the opening of the acetabular cup component appears as an ellipse. Software systems, such as the Surgeon's Checklist® Hip system from Radlink, Inc. of El Segundo, CA, analyze the fluoroscopic images, including reference lines added by the surgeon and the elliptical opening of the acetabular cup, and calculate inclination and anteversion. If the calculated values are within the desired ranges, the components may be left in place or the trial components may be replaced with final components matching the position and/or orientation of the trial components. If the values are not within the desired ranges, the components (trial or final) may be repositioned and/or re-oriented and/or different components (trial or final) may be evaluated.

Nonetheless, determining accurate measurements of the cup component via the fluoroscopic PA and AP images is problematic due to issues associated with magnification and distortion in fluoroscopic images, confusion about orientation of the image relative to the patient, and a general lack of knowledge about the patient's unique three-dimensional structure. Inaccuracies in the measurements, moreover, can lead to inaccuracies in implant positioning.

### SUMMARY OF THE INVENTION

According to one aspect of the invention, a computer-implemented method is provided. The method comprises:
receiving pre-operative volume data for at least a portion of a patient's anatomy, the volume data including a plurality of volume elements (voxels) having assigned values associated with radiographic density of the portion of the patient's anatomy;
generating, by at least one processor, a three-dimensional (3D) model of the patient's anatomy from the volume data;
placing a 3D model of an implant at a planned position or orientation at least partially within the 3D model of the patient's anatomy to create a 3D post-implantation model of the patient's anatomy;
following the placing, translating, by the one or more processors, the 3D post-implantation model of the patient's anatomy into a plurality of voxels of planned post-implantation volume data that correspond to the portion of the model of the implant positioned within the planned position or orientation, the translation including changing the assigned values of the plurality of voxels in the volume data to one or more new values in the planned post-implantation volume data;
creating, by the one or more processors, one or more predicted synthetic digital radiographs (DRs) from the planned post-implantation volume data; and
presenting the one or more predicted synthetic DRs on a display, wherein the one or more predicted synthetic DRs show an image of the implant at the planned positions and/or orientations within the patient's anatomy.

Briefly, the present disclosure relates to computer-based systems and methods for generating a surgical plan from which one or more predictive synthetic digital radiographs (DRs) can be generated that show one or more prosthetic components at planned positions and/or orientations relative to patient anatomy. The synthetic DRs with radiograph images of prosthetic component models placed relative to the patient's anatomy in the planned manner may be generated before the surgical procedure and before physical prosthetic components are implanted in the patient. The systems and methods may also support comparisons between the one or more of the predictive synthetic DRs and image data obtained during surgery, e.g., to evaluate whether the physical prosthetic components are implanted as planned.

For example, the systems and methods may generate a surgical plan for performing total hip arthroplasty (THA) on a patient. The systems and methods may receive pre-operative, volume data for at least a portion of a patient's anatomy, e.g., the patient's pelvis and/or femur. Exemplary volume data may be generated using Computed Tomography (CT) or Magnetic Resonance Imaging (MRI). The volume data may be arranged on a Cartesian voxel grid.

In some cases, the pre-operative volume data may include actual CT data or predicted CT data. Predicted CT data may be derived, for example, from stereoradiographic imaging such as EOS® biplanar X-ray imaging using appropriate 3D reconstruction techniques.

The volume data may be used to generate image data, such as axial, coronal or sagittal anatomical slices or reconstructions, from which the three-dimensional model of the patient's anatomy can be created.

The systems and methods may generate a three-dimensional (3D) model of the portion of the patient's anatomy from the volume data. In some embodiments, the systems and methods may define separate sub-volumes for different elements or portions of the patient's anatomy, such as one sub-volume for the pelvis and another sub-volume for the femur on the operative side. The systems and methods may include a library of 3D models of prosthetic hip components, such as femoral hip stems, femoral heads, cup components, and liners. The systems and methods may include a planning tool, which may be utilized to select particular hip component models from the library and to place them on or in the 3D pelvis and/or femur model. The component models may be placed on and/or integrated into the 3D models of the pelvis and femur at planned positions and/or in planned orientations, e.g., to achieve one or more goals of the surgery, such as planned inclination and/or anteversion of the cup component and/or restoration or changes to the patient's leg length, offset, and/or anterior-posterior (AP) position.

In some embodiments, one or more models of surgical tools, such as reamers or impactors, may also be placed within the 3D model at planned positions and/or orientations for the tools. Volume or voxel elements (voxels) corresponding to the tool models may be assigned values representing the radiographic density of the material from which the physical tools are made, such as metal or polymer materials.

The systems and methods may replace voxels from the volume data for portions of the femur and femoral head with values for the prosthetic stem and head. The systems and methods also may replace voxels from the volume data for the acetabulum (to be removed, e.g., through reaming) with values for the cup component of the planned size, position and orientation. With the models of the prosthetic components placed in the planned positions and/or orientations, the femur sub-volume may be joined to the pelvis sub-volume, e.g., the model of the prosthetic femoral head may be centered in the model of the prosthetic acetabular cup component, with the femur placed in an anatomically corrected orientation, e.g., straight down relative to the pelvis.

In some embodiments, synthetic imaging of the femur may be simplified. Synthetic DRs may be generated separately for the femur and the pelvis and then joined together to create a synthetic DR as opposed to joining them together at the 3D model and volume data stage. In practice this can allow for the 3D volume and various synthetic DRs for different orientations of the pelvis to be generated as needed (with reduced complication through focus only on the pelvis without 3D volume data of the femur) while only a single or few synthetic DRs of the femur are used to join with the various pelvic synthetic DRs. The predictive image of the reconstructed femur could be prepared from an appropriate angle (e.g., perpendicular to the condylar plane of the femur) and that single synthetic DR of the reconstructed position would be appropriate for joining onto several pelvis images at various orientations. This can prove advantageous in situations such as anterior hip surgery, where the surgeon is able to control the position of the femur fairly well since the leg is typically positioned with the foot in a boot. Accordingly, it is relatively easy to position and maintain the leg with a straight on view of the femur (essentially perpendicular to the condylar plane). It is typically more difficult to maintain a consistent position and orientation for the pelvis during surgery, however. The ability to separately generate synthetic DRs for those anatomical structures or portions thereof can therefore increase efficiency and reduce computational demands by limiting the creation of new synthetic DR images only to the anatomical portions that may be changing position or orientation.

The following steps can be used to align and join, for example, a pelvic synthetic DR and a femoral synthetic DR. Pelvic synthetic DRs can be generated relative to any pelvic coordinate system (COS) but the two most common COSs are the Function Pelvic COS, meaning how the patient is positioned in a CT scanner in the raw CT coordinate space, or relative to the APPlane COS. Those synthetic DRs may be generated as described above, with the femur masked out along with any tools or implants related to a planned surgery and present on the pelvis excluded or included as desired.

The synthetic DRs of the femur can be generated relative to any femoral COS with the two most common being the condylar plane COS (two points (medial and lateral) on the distal femur and the third point being the base of the femoral neck) or the anteversion plane COS (the three points of which would be the prosthetic head center, the base of the neck of the prosthesis, and the center of the knee). The femoral synthetic DRs can be generated with the pelvis masked out and any tools, devices, implants, or other machinations related to a planned surgery and present on the femur included or excluded as desired.

The two synthetic DRs can then be joined by having the center of rotation of the acetabular component and the center of rotation of the femoral component coincide. Rotationally, the femur can be positioned straight down so that on the femoral side a line between the femoral center and the center of the knee is perpendicular to, on the pelvic side, a line connecting the right and left anterior superior iliac spines and parallel to the APP COS plane or in fact, the plane of the pelvis x-ray.

As noted, it may be advantageous to connect a single preferred femoral image to a variety of pelvis images. In any case, those images, may be connected at the planned centers of rotation with the two image planes coincident with each other, and the femoral axis point “down” from an adduction/abduction point of view.

The surgical plan may specify one or more prosthetic components to be implanted in the patient's body as well as the components' positions and/or orientations. For example, the surgical plan may specify a particular cup component and its position at a patient's acetabulum, e.g., depth, and an orientation within the acetabulum. The plan may also include the shape of the cup bed to receive the cup component. For example, the surgical plan may specify a particular volume or portion of bone in and/or around the patient's acetabulum that is to be removed, e.g., reamed, in order to receive the acetabular cup component. It may also specify a planned position and/or orientation of prosthetic stem component at the patient's femur.

The systems and methods may construct one or more predicted volume, e.g., CT, data based on the one or more 3D models as modified to include the 3D models of the prosthetic components placed at the planned positions and/or orientations. As noted, the systems and methods may identify voxels in the original volume, e.g., CT, data that correspond to locations where the prosthetic components are placed on the pelvis model, and these voxels may be replaced with values representing the prosthetic component, e.g., a value representing the radiographic density of the cup component.

The new values may match the radiographic density of the material from which the implant is made, e.g., titanium, or may be selected to optimize visualization in the resulting synthetic image. For example, the actual radiographic density of titanium can lead to difficulty in visualizing details in radiographic images of the implant so synthetic DRs of a titanium implant may be created with slightly altered predicted values to better show details in the synthetic DR.

Even though it is created before the surgical procedure is performed, the predicted volume data may thus simulate a post-surgery CT in which the prosthetic components were implanted at the planned positions and/or orientations. The systems and methods may generate one or more synthetic DRs from the predicted volume data. Because the synthetic DRs are generated from the predicted volume data (not the original volume, e.g., CT, data), these predictive synthetic DRs show the prosthetic components at the planned positions and/or orientations. The systems and methods may generate the predictive synthetic DRs to match intended or expected intra-operative fluoroscopic images.

The generation of synthetic DRs may involve specifying the position of a virtual x-ray source and detector and defining one or more x-ray beam angles relative to the volume data.

The predictive synthetic DRs, however, may include far greater information, e.g., a larger field of view, than could be obtained intra-operatively, e.g., using a fluoroscopic imaging machine. The systems and methods may designate the portion of the predictive synthetic DR that is viewable by a fluoroscopic imaging machine. For example, a rectangle, circle, or other boundary may be added to the synthetic DR. Inside the boundary is the view that can be generated by the fluoroscopic machine, while outside the boundary is additional information that could not be created by the fluoroscopic machine. In some embodiments, the systems and methods may combine, e.g., stitch together, two or more predictive synthetic DRs to create a composite synthetic DR, such as a predictive synthetic DR of the preferred plan of the pelvis and cup component from a preferred orientation, with a predictive synthetic DR of the preferred plan of the femur and femoral component from a different preferred orientation. In some embodiments, the predictive synthetic DRs may be included in the surgical plan.

In various embodiments, graphical affordances, such as reference marks or error gauges, may be included in one or more predictive synthetic DRs in order to allow a user to quickly identify and quantify any differences between planned and actual placement or orientation of the patient's anatomy or prosthetic components (e.g., implants). For example, reference marks around the acetabular cup component in a planned THA surgery can be included on the predictive synthetic DR that may show various degrees of anteversion and higher or inclination of the cup, higher or lower, deviating from the planned orientation. Similarly, as the femur is prepositioned relative to the pelvis, reference marks such as horizontal and longitudinal rulers superimposed on the synthetic DR images of the femur can be included to allow for quick quantification of deviations from the planned change in leg length and offset. For example, when the synthetic DR with reference marks is overlaid onto the intra-operative fluoroscopic image and the fluoroscopic does not exactly match up with the synthetic DDR, then the reference marks can readily illustrate how far off the actual implant is relative to the plan. Accordingly, a user in the operating room can readily identify an exact deviation in length or offset without the need for calculations on the images and without the need for any image recognition or analysis systems.

During surgery, physical prosthetic components that correspond to the planned components may be implanted at the patient's hip. For example, the physical components may be implanted at the planned positions and/or and orientations. During the procedure, intra-operative radiographs may be taken of the patient. For example, a C-arm type fluoroscopy machine may be used to generate intra-operative radiographs during or after the physical prosthetic components have been implanted. The radiographs may be presented on a display screen in the operating room for evaluation by the surgeon. In some embodiments, the systems and methods may present the synthetic DRs side-by-side with the intra-operative radiographs from the fluoroscopy machine and/or may superimpose one or more synthetic DRs onto the intra-operative radiographs. The systems and methods may resize and/or rescale the predictive synthetic DRs to match the intra-operative radiographs. In some embodiments, intra-operative fluoroscopic images may be automatically scaled and oriented to correspond to relevant synthetic DRs and then overlayed thereon. By comparing the intra-operative radiograph with the predictive synthetic DR, regardless of the method of comparison, the surgeon may determine whether or not the physical prosthetic components are placed in the planned positions and/or orientations.

In some embodiments, the systems and methods may overlay one or more of the predictive synthetic DRs onto intra-operative x-ray images to facilitate direct visual comparison between planned and achieved component placement.

In certain embodiments, the synthetic DRs may be presented on a mixed reality head-mounted device (MR-HMD) or other computing device, which may superimpose or compare the synthetic image with intra-operative radiographs.

In some embodiments, the systems and methods may analyze an intra-operative radiograph and determine settings, such as locations of virtual x-ray source and virtual detector, x-ray beam angles, etc. for use in generating a predictive synthetic DR in a live manner during the surgical procedure. In some embodiments, an MR HMD (or other computing device) may automatically analyze an intraoperative x-ray image and choose a pre-generated predictive synthetic DR from the same angle and other characteristics for comparison. In other embodiments, the MR HMD may analyze the intraoperative x-ray image and direct the predictive synthetic DR generator 408 to generate a predictive synthetic DR during the surgical procedure using the same characteristics (e.g., center, angle, x-ray source to receiver distance, position of the body relative to the x-ray source to receiver distance) as the intraoperatively acquired image. The MR HMD (or other computing device) may utilize a 2D/3D match algorithm, such as described Steppacher S D, Tannast M, Zheng G, Zhang X, Kowal J, Anderson S E, Siebenrock K A, Murphy S B. Validation of a new method for determination of cup orientation in THA. Journal of Orthopedic Research. 2009 December; 27 (12): 1583-8 and in Guoyan Zheng, Xuan Zhang, Simon D. Steppacher, Stephen B. Murphy, Klaus A. Siebenrock, Moritz Tannast, HipMatch: An object-oriented cross-platform program for accurate determination of cup orientation using 2D-3D registration of single standard X-ray radiograph and a CT volume, Computer Methods and Programs in Biomedicine, Volume 95, Issue 3, 2009, Pages 236-248, ISSN 0169-2607.

In some embodiments, the systems and methods may generate one or more predictive synthetic DRs from the predicted volume data during the surgical procedure. Specifically, the systems and methods may generate the predictive synthetic DR such that the image plane of the synthetic DR matches the image plane of the intra-operative radiograph, e.g., as taken with a C-arm fluoroscopy machine. The systems and methods may analyze an intra-operative radiograph and determine the angle at which the intra-operative radiograph was taken. For example, the systems and methods may generate a synthetic DR using the C-arm angle settings used to take the intra-operative radiographs. The systems and methods may display the matching, synthetic DR side-by-side or superimposed on the intra-operative radiograph or in other manners that lend to comparing the synthetic radiograph with the intra-operative radiograph.

In various embodiments, intra-operative radiographs or other images may be loaded into an image analysis application and automatically scaled and superimposed over predicted synthetic DRs of the invention.

As noted, in some embodiments, the systems and methods may include a Mixed Reality (MR) Head-mounted device (HMD) that may be worn by the surgeon during the surgical procedure. The MR-HMD may display the predictive synthetic DRs. The MR-HMD may be programmed to automatically superimpose the predictive synthetic DRs on intraoperative x-ray images and to perform automated comparisons and reported analytics. The MR-HMD may also display one or more holograms of the surgical procedure.

The physical prosthetic components may be implanted at positions and/or orientations that differ somewhat from the planned positions and/or orientations. In addition, in some cases, the surgeon may use different physical components rather than the planned components. The systems and methods can update the surgical plan and can generate updated predicted synthetic DRs, which can be presented during the surgical procedure in real time.

In certain embodiments, the pre-operative volume data can include computed tomography (CT) data or predicted computed tomography (CT) data. In some embodiments, the pre-operative volume data can be derived from magnetic resonance imaging that may be altered to match the contrast of CT data, thereby providing predicted CT data. Furthermore, the predicted CT data may be derived from stereoradiographic imaging such as EOS® biplanar X-ray imaging or traditional radiographs. Methods may include repositioning the 3D model of the patient's anatomy or one or more portions thereof relative to one or more references. In some embodiments, the patient's anatomy for which the predicted synthetic DRs are being created can include a femur and the one or more references can comprise a pelvis. For example, a 3D model of the femur may be repositioned such that an axis between a planned prosthetic femoral head center to a knee center is perpendicular to a medial lateral axis of the pelvis. In some embodiments, a 3D model of the femur can be repositioned such that an axis defined by a planned prosthetic femoral head center to a knee center according to the pelvic tilt (supine from the plan or a number entered from measurement of a standing view. In certain embodiments, the patient's anatomy can include a femur and the one or more references comprise a planned acetabular component for a planned total hip arthroplasty (THA) such that the 3D model of the femur is repositioned relative to the planned acetabular component of the hip replacement. Such repositioning can be used to compensate for any defects in positioning of the patient when the original volume data was obtained (e.g., through CT scan) or to compensate or correct any anatomical defects that the surgeon may wish to address during surgery (e.g., adjusting leg length and/or offset along with rotation of the femur after prosthetic reconstruction compared to femoral rotation prior to reconstruction).

Aspects of the invention may include a computer system comprising a processor in communication with a non-transitory, tangible memory storing instructions that, when executed by the processor, perform any of the computer-implemented methods described herein.

### BRIEF DESCRIPTION OF THE DRA WINGS

The description below refers to the accompanying drawings, of which:

FIG. 1 is an exploded view of an example hip implant;

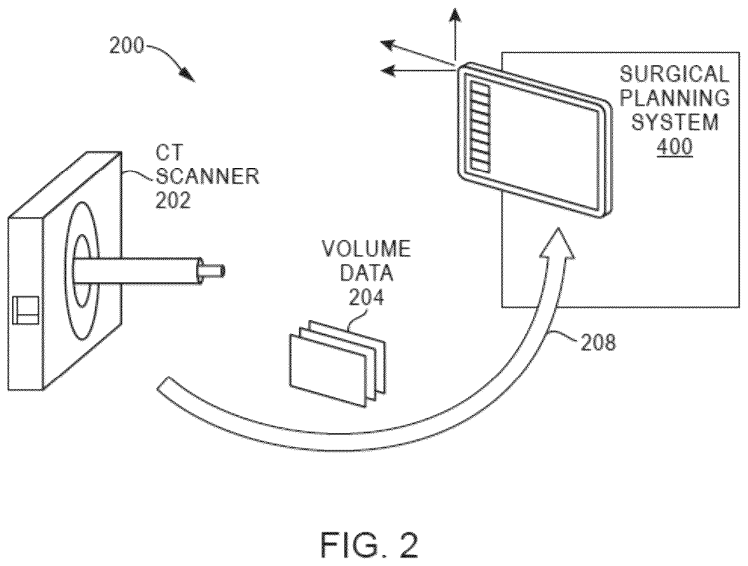

FIG. 2 is a schematic illustration of an example data capture system in accordance with one or more embodiments;

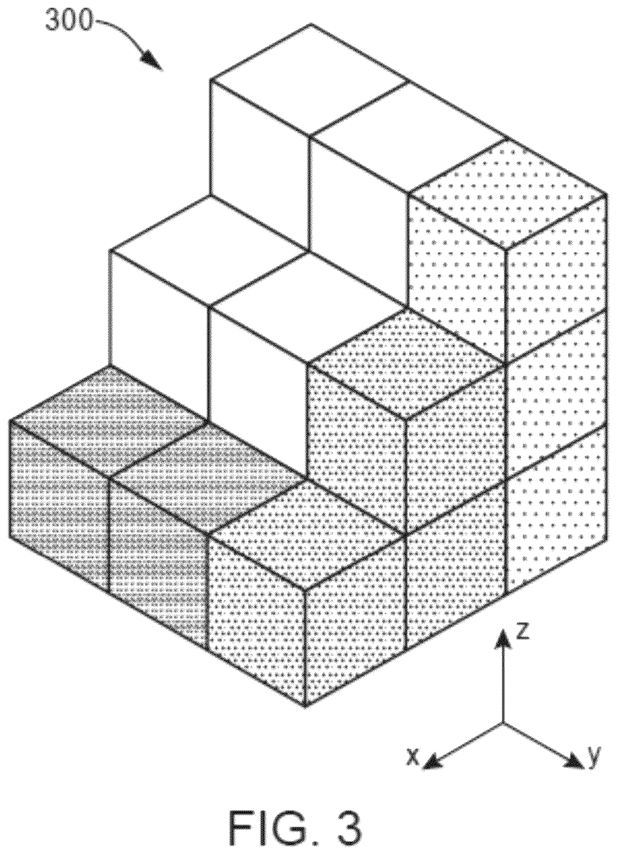

FIG. 3 is a schematic illustration of an example three-dimensional voxel array in accordance with one or more embodiments;

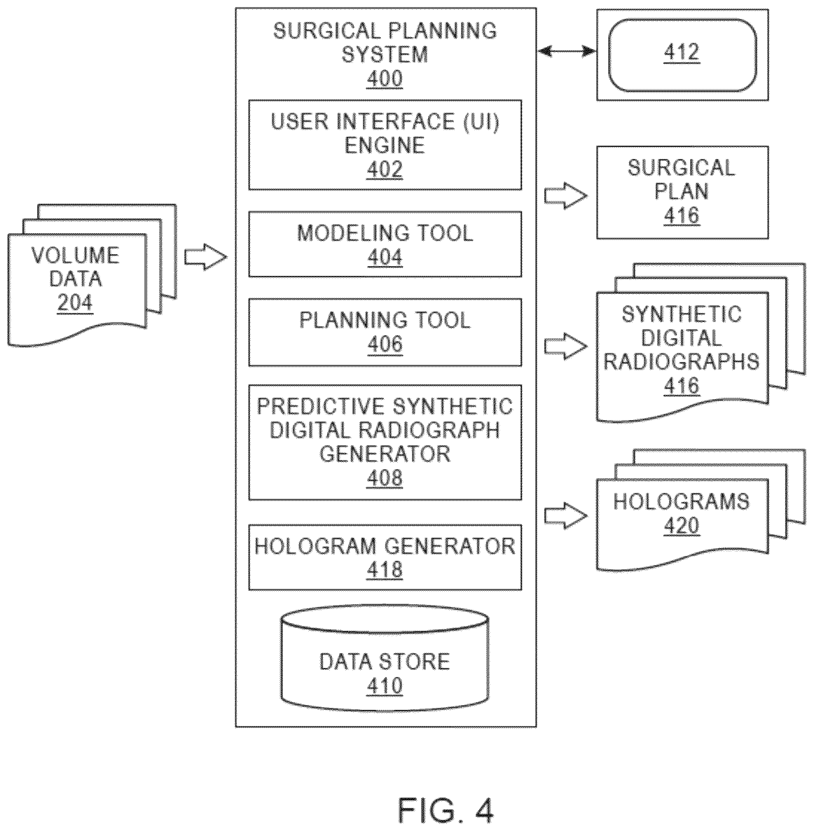

FIG. 4 is a schematic illustration of an example of the surgical planning system in accordance with one or more embodiments;

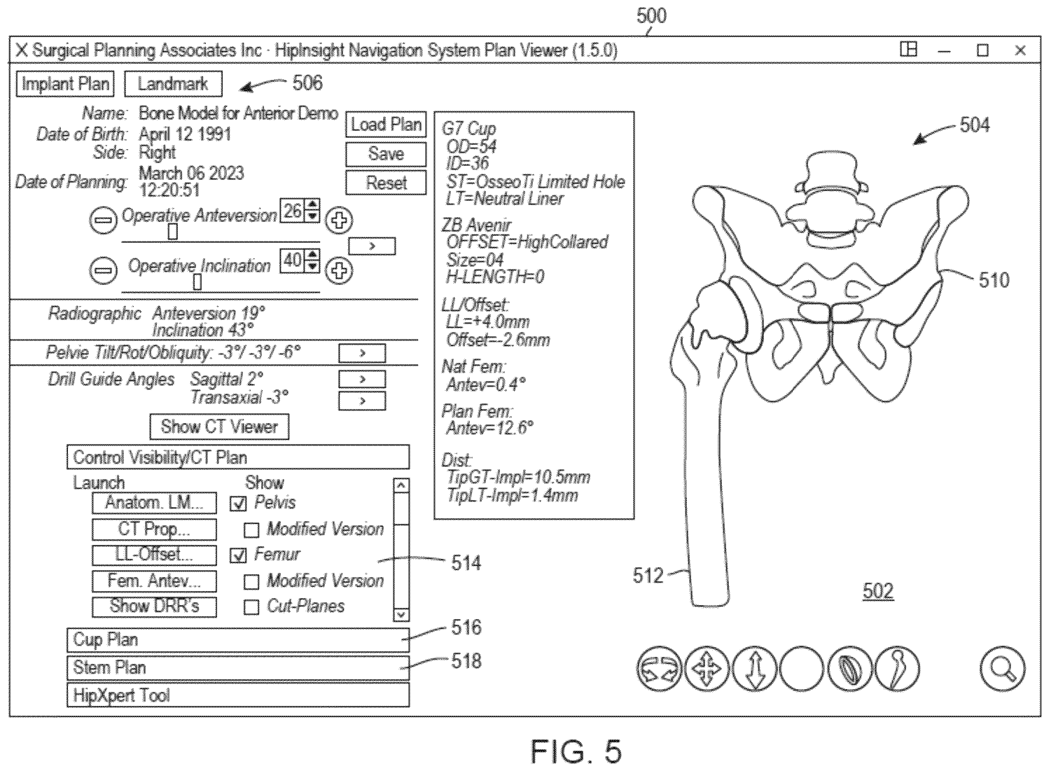

FIG. 5 is an illustration of an example planning window in accordance with one or more embodiments;

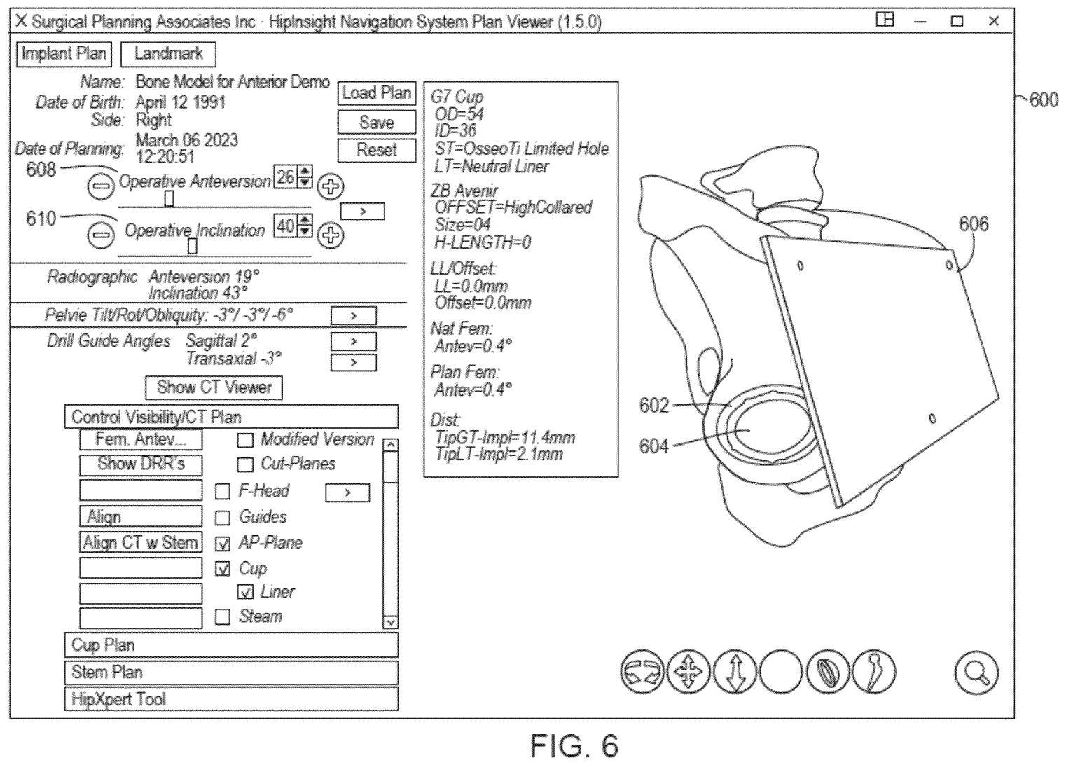

FIG. 6 is an illustration of another example planning window in accordance with one or more embodiments;

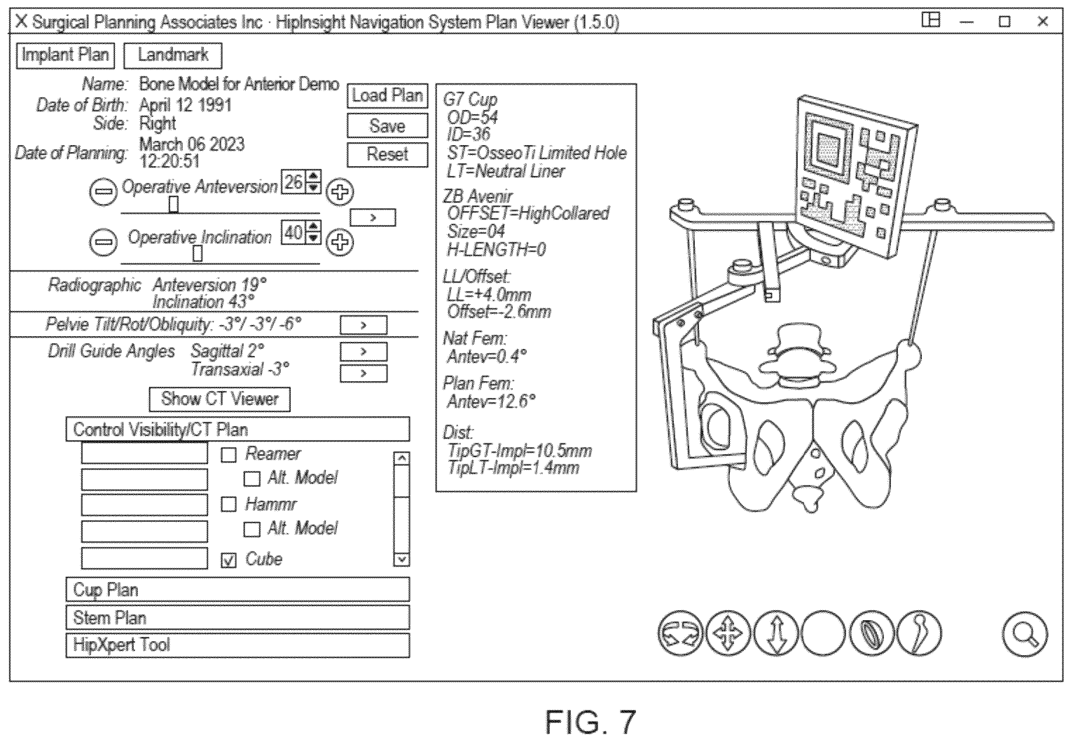

FIG. 7 is an illustration of another example planning window in accordance with one or more embodiments;

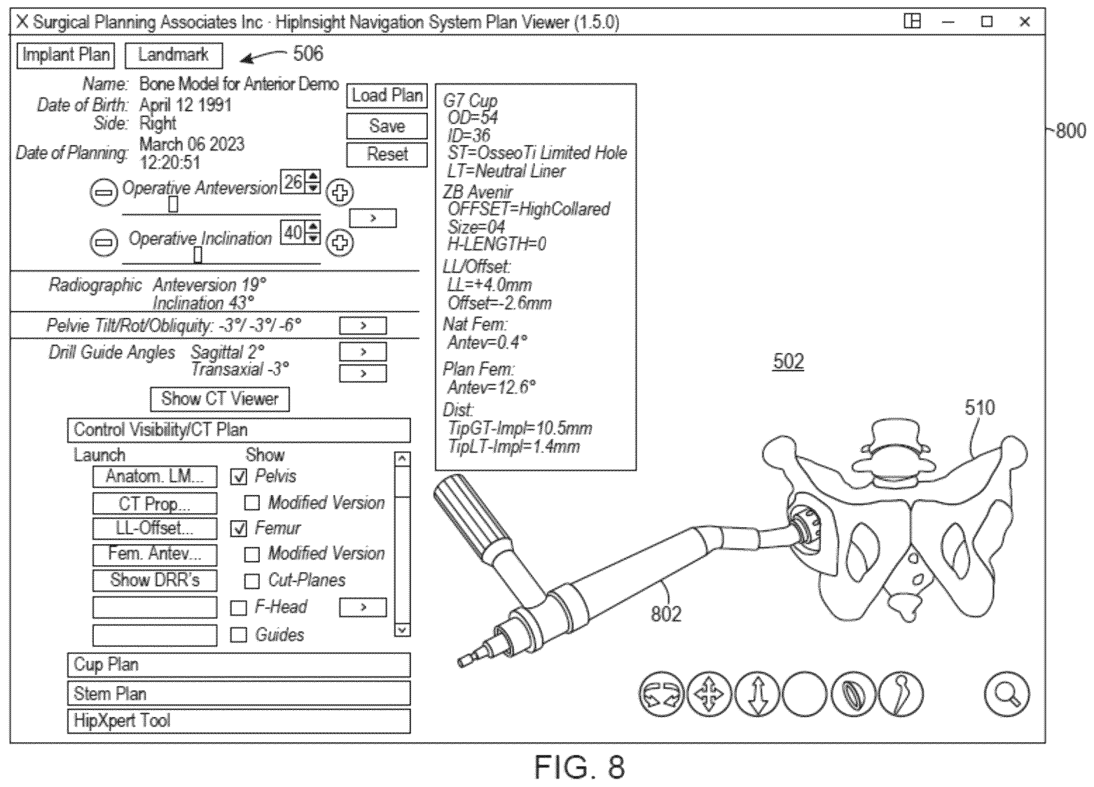

FIG. 8 is an illustration of another example planning window in accordance with one or more embodiments;

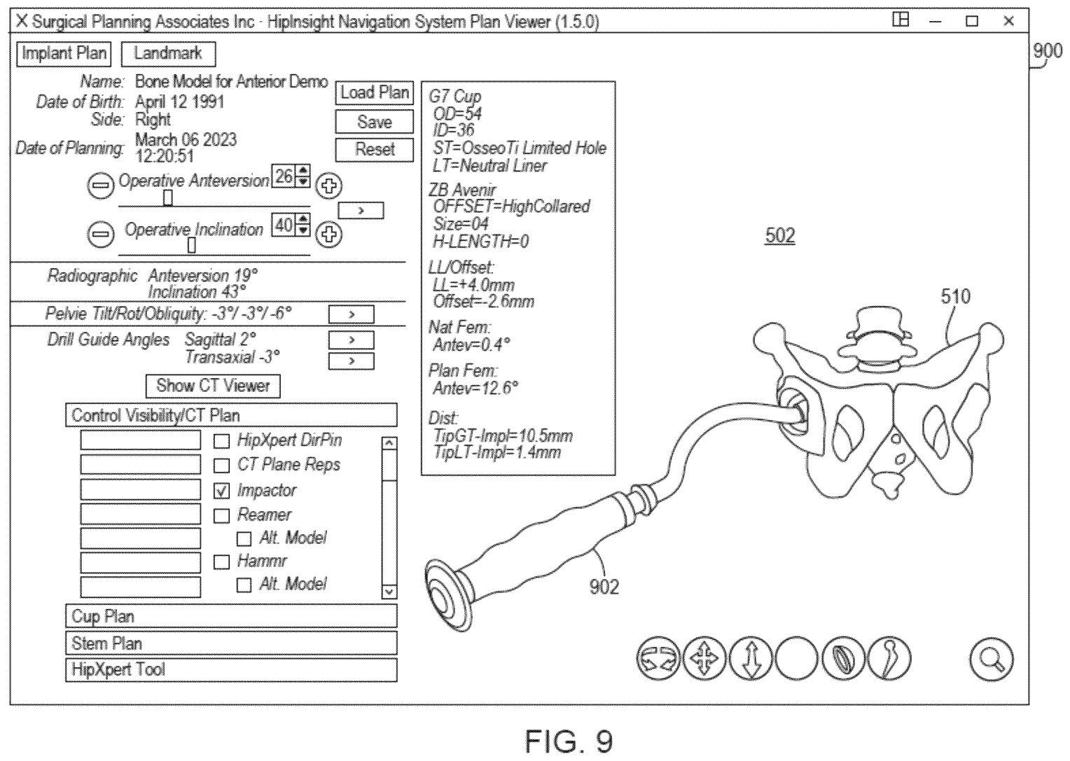

FIG. 9 is an illustration of another example planning window in accordance with one or more embodiments;

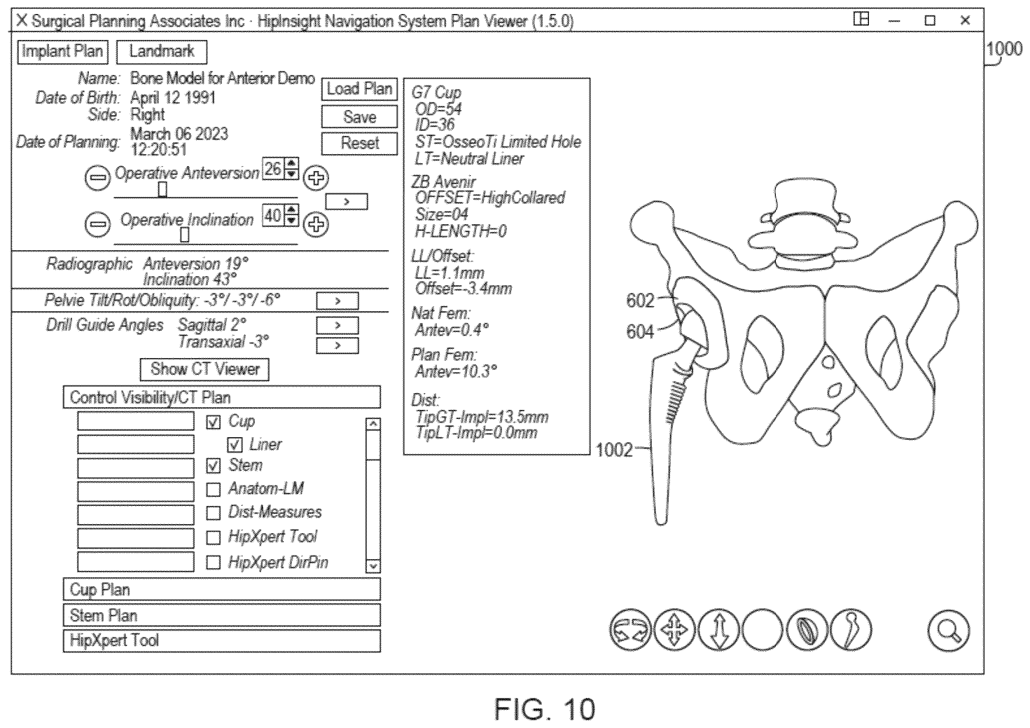

FIG. 10 is an illustration of another example planning window in accordance with one or more embodiments;

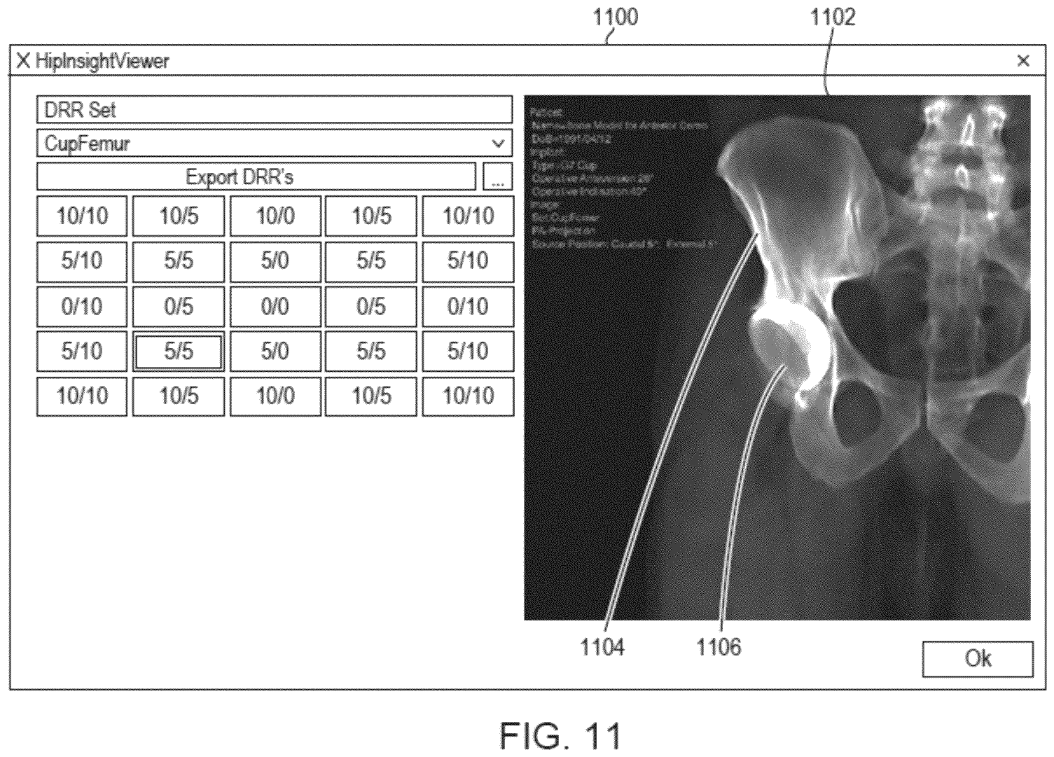

FIG. 11 is an illustration of an example planning window presenting a synthetic DR 1102 as generated in accordance with one or more embodiments;

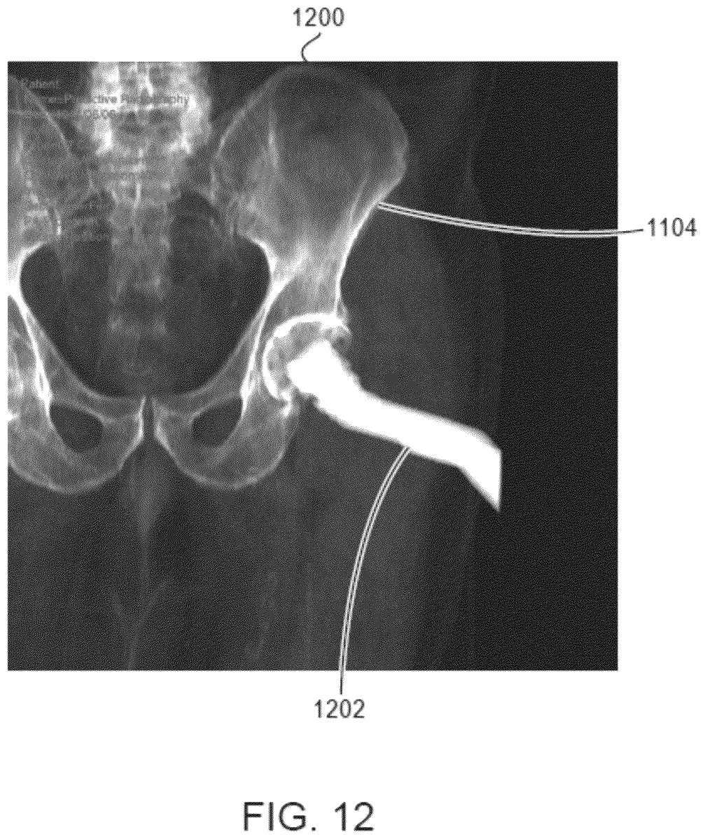

FIG. 12 is an illustration of an example synthetic digital radiograph (DR) in accordance with one or more embodiments;

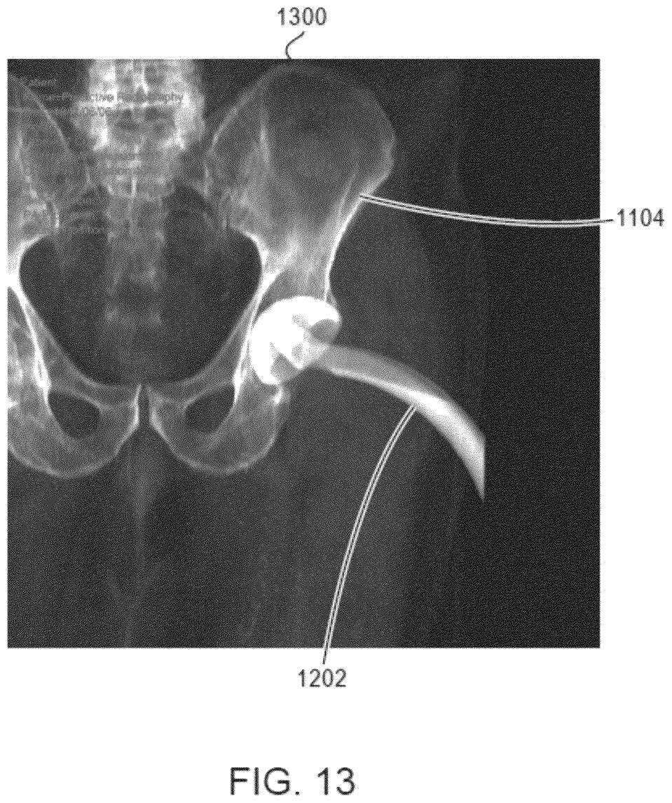

FIG. 13 is an illustration of another example synthetic DR in accordance with one or more embodiments;

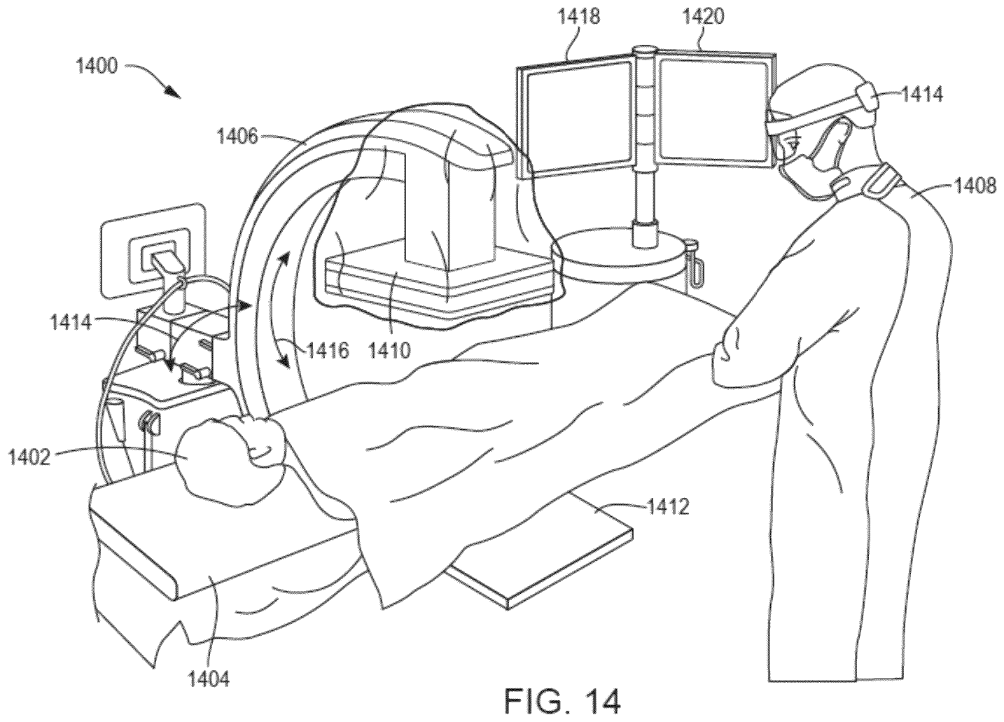

FIG. 14 is a schematic illustration of an operating room in accordance with one or more embodiments;

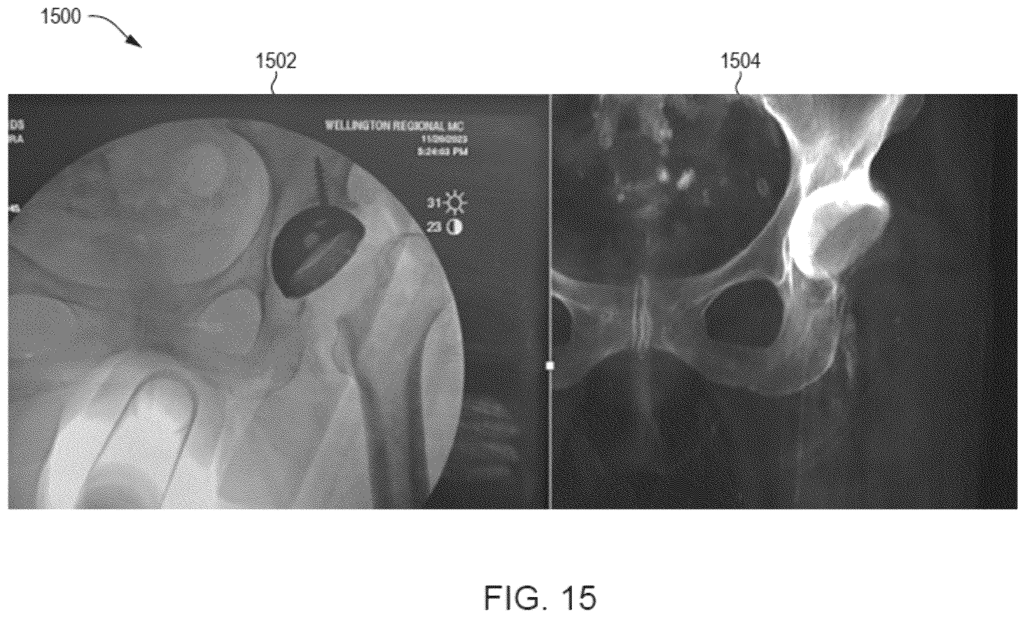

FIG. 15 is an illustration of an example intra-operative display in accordance with one or more embodiments;

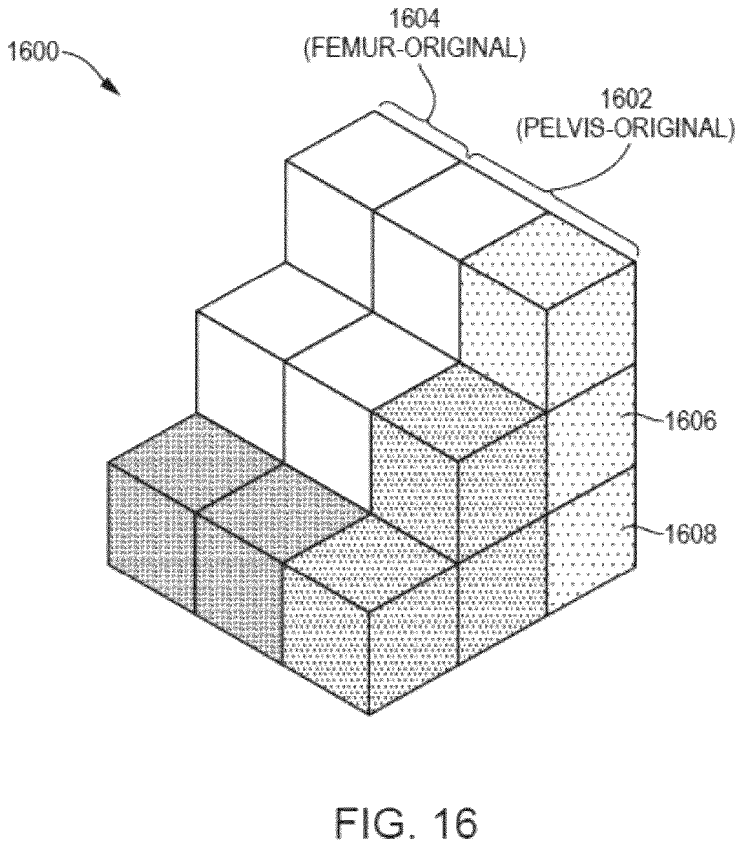

FIG. 16 is a schematic illustration of an example three-dimensional voxel array in accordance with one or more embodiments;

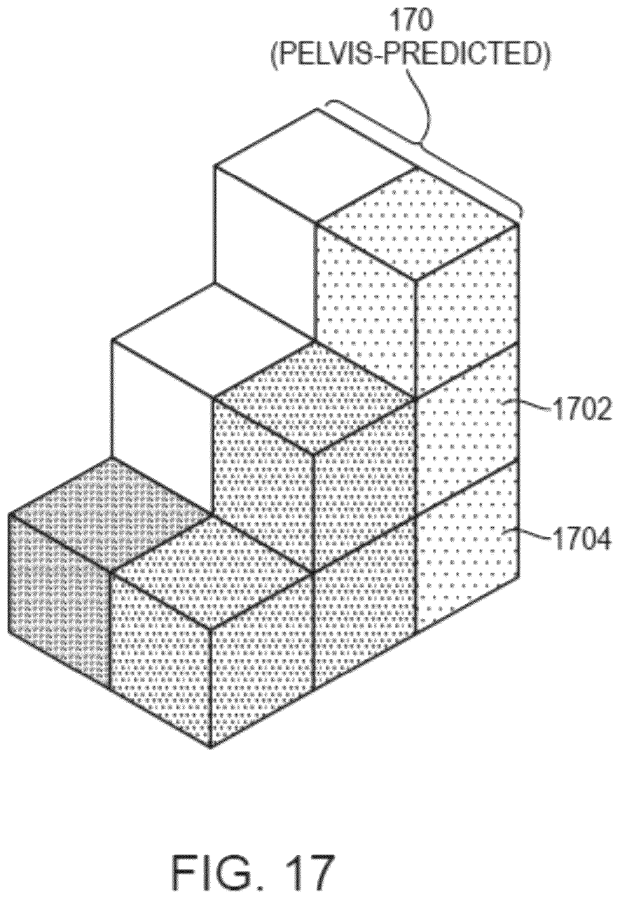

FIG. 17 is a schematic illustration of an example three-dimensional voxel array in accordance with one or more embodiments;

FIG. 18 is a schematic illustration of a planning window 1800 in accordance with one or more embodiments;

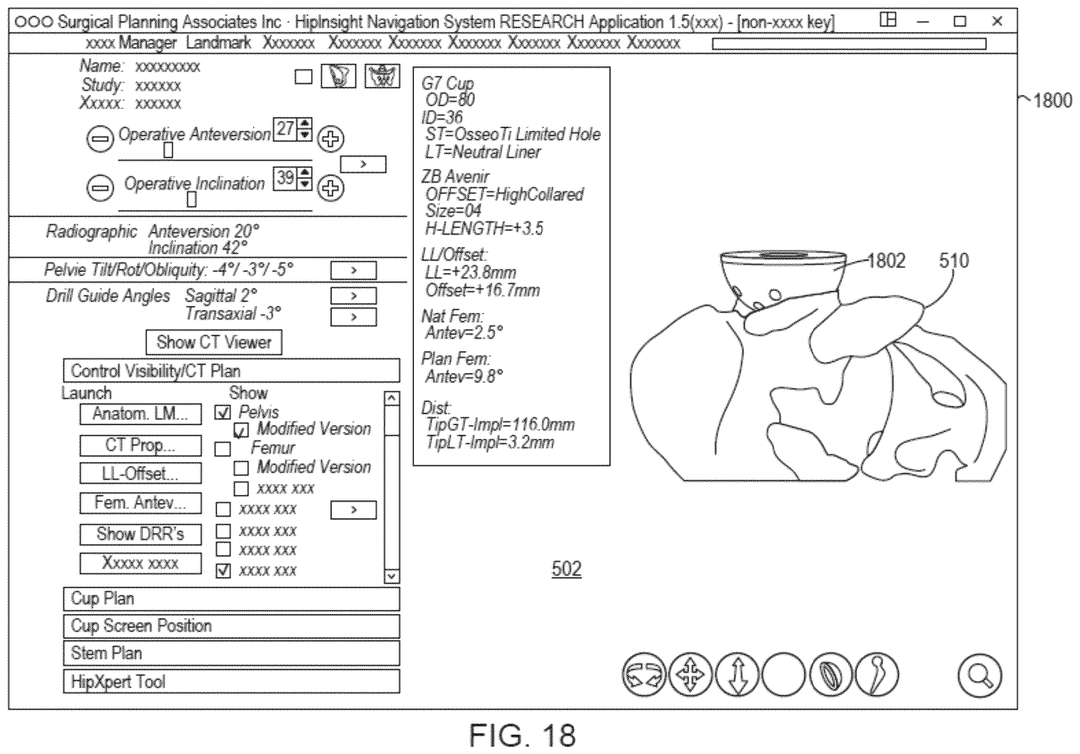

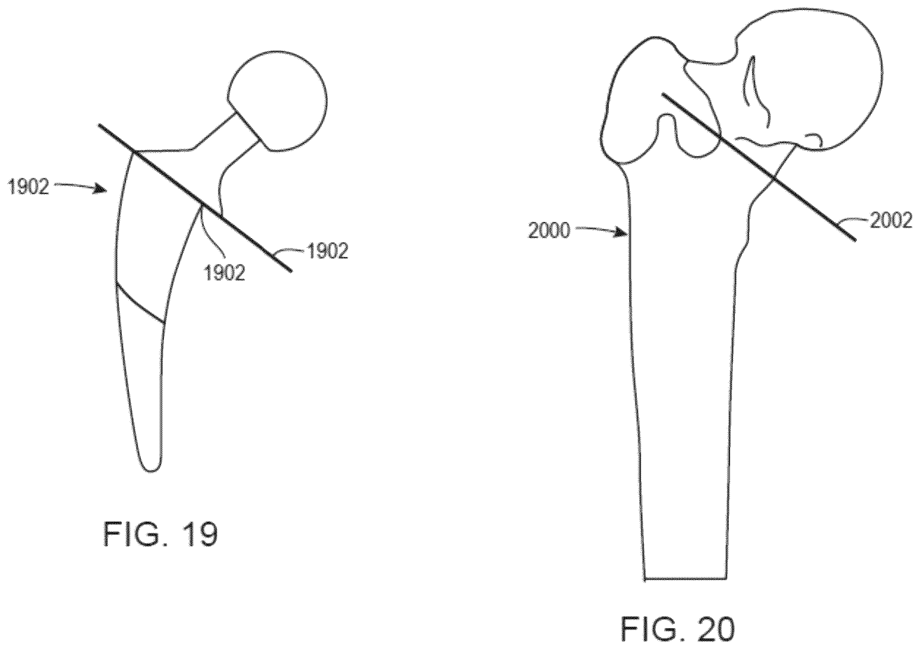

FIG. 19 is a schematic illustration of a front view of an example femoral component in accordance with one or more embodiments;

FIG. 20 is a schematic illustration of a side view of an example femur model in accordance with one or more embodiments;

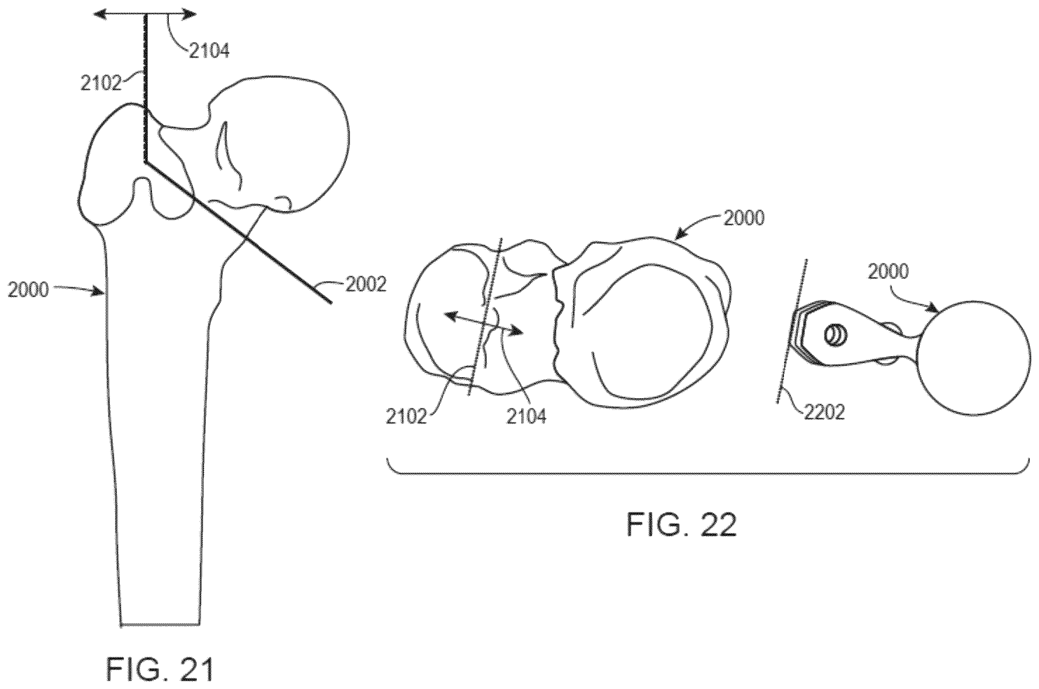

FIG. 21 is a schematic illustration of another front view of the femur model in accordance with one or more embodiments; and

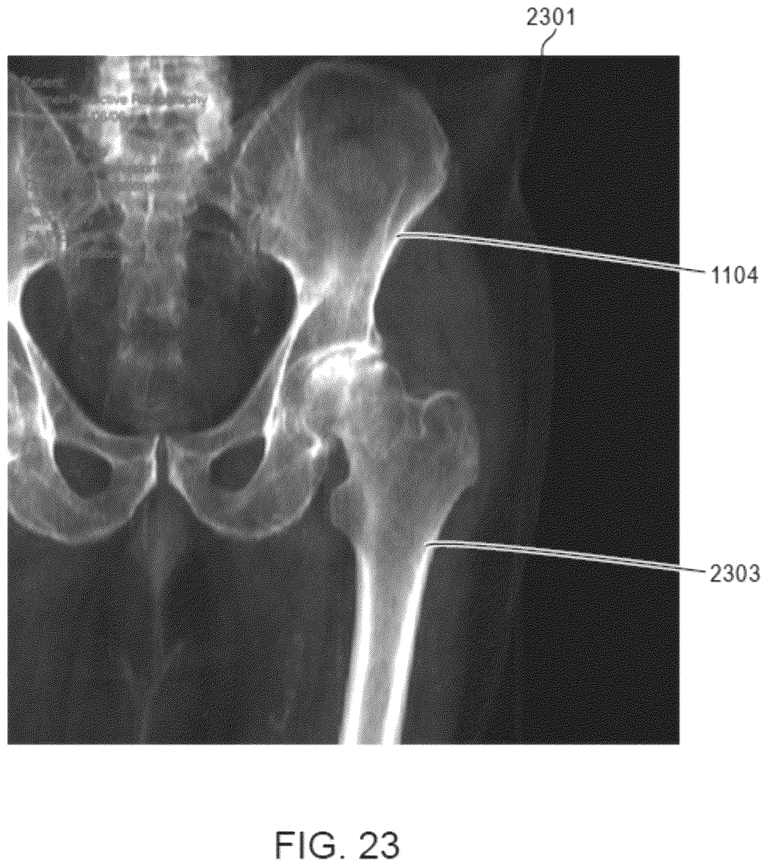

FIG. 22 is a schematic illustration of a top view of the femur model and a top view of the femoral component 1900 in accordance with one or more embodiments.

FIG. 23 shows an exemplary unmodified synthetic DR in accordance with one or more embodiments.

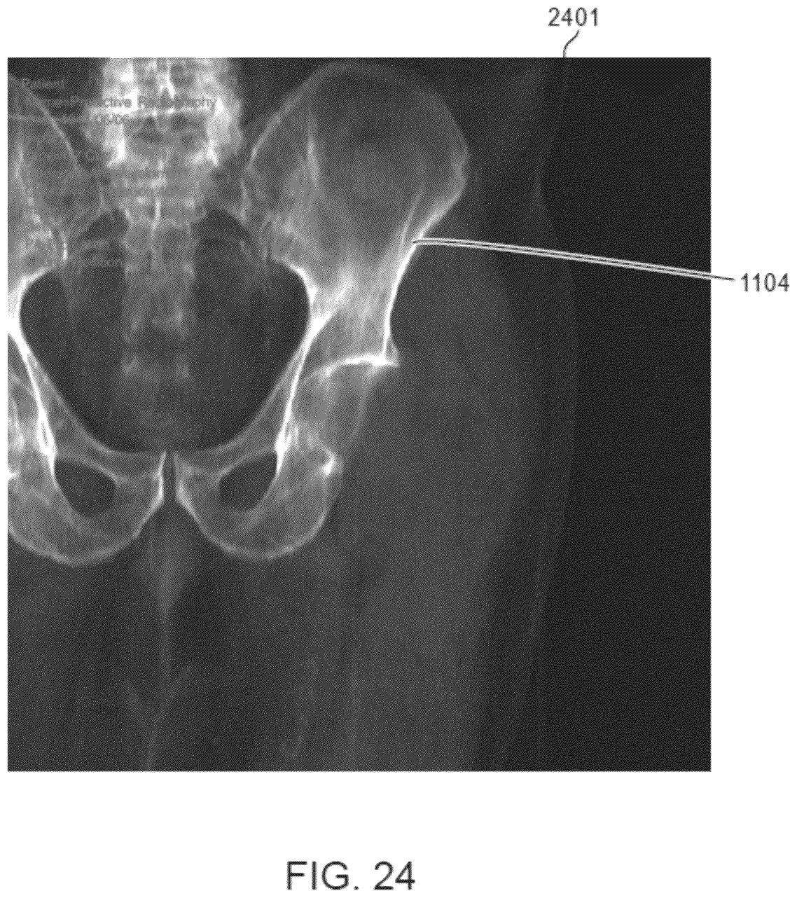

FIG. 24 shows an exemplary synthetic DR modified to remove the appearance of the femur in accordance with one or more embodiments.

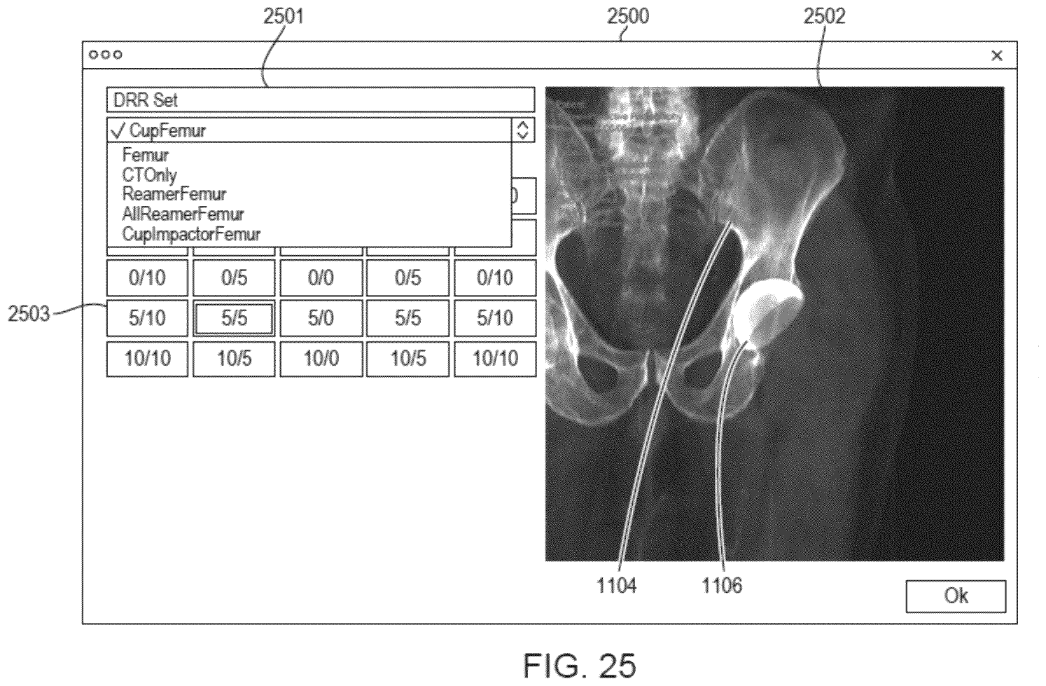

FIG. 25 shows an exemplary planning window for generating one or more synthetic DRs including a drop-down menu with available modified views in accordance with one or more embodiments.

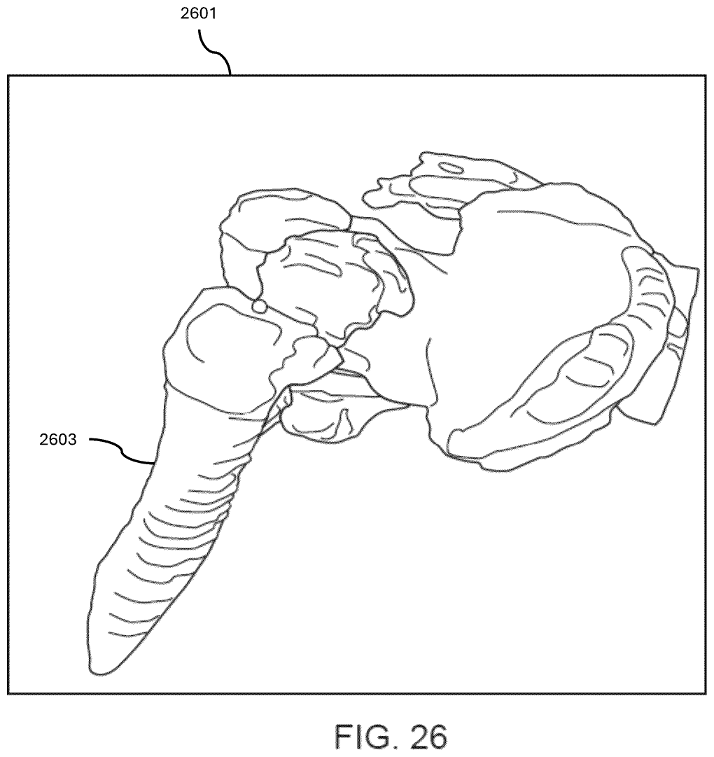

FIG. 26 shows a CT scan of an example of a severely malpositioned femur.

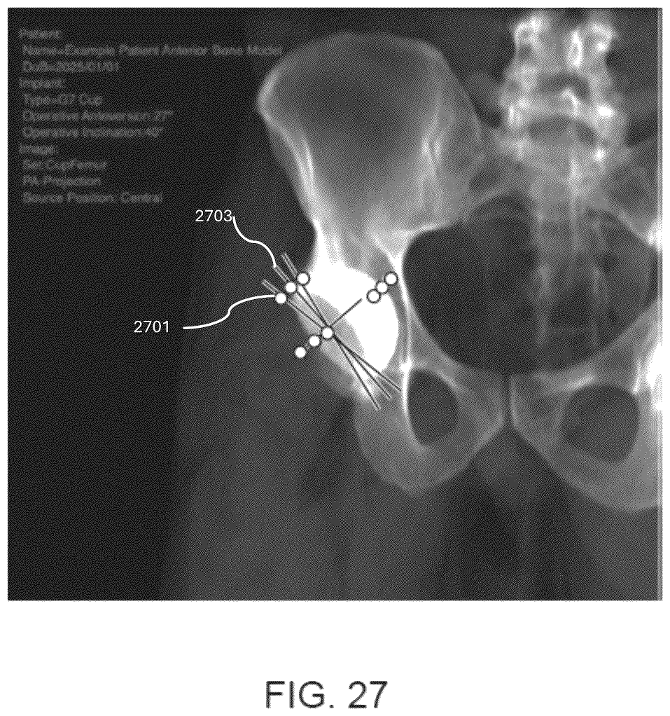

FIG. 27 shows an example of a synthetic DR including error gauges in accordance with one or more embodiments.

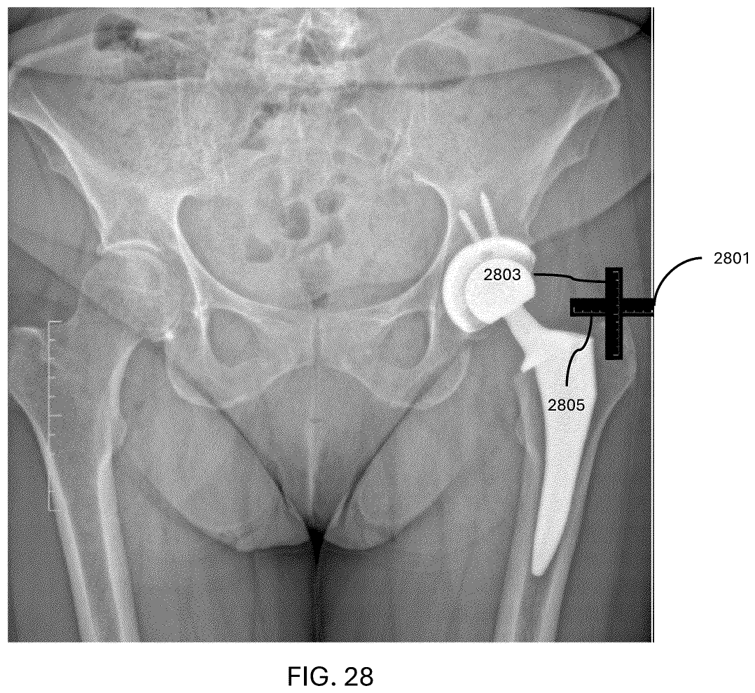

FIG. 28 shows an example of a synthetic DR including reference marks or rules for length and offset of the femur in accordance with one or more embodiments.

### DETAILED DESCRIPTION OF ILLUSTRATIVE EMBODIMENTS

The present disclosure addresses the need for accurate intra-operative assessment of prosthetic component placement during orthopedic procedures, particularly total hip arthroplasty (THA). Conventional imaging methods, such as intra-operative fluoroscopy, are limited by distortion, magnification effects, and a lack of detailed three-dimensional anatomical context. These limitations may lead to inaccuracies in the positioning of prosthetic implants, potentially resulting in complications and revision surgeries.

It is therefore an object of the present disclosure to provide a computer-implemented method for generating radiographic images that reflect a planned post-implantation configuration of a prosthetic component within a patient's anatomy, based on pre-operative volume data, in order to enable comparison with intra-operative radiographic images and support accurate surgical decision-making.

The disclosed method enables the generation of predictive synthetic digital radiographs (DRs) that simulate the radiographic appearance of a planned implant placement within a patient's anatomy. The method is based on pre-operative volume data, such as computed tomography (CT) data, in which each voxel includes a value corresponding to radiographic density.

A three-dimensional model of the relevant anatomical structures is generated from the volume data. A model of an implant is placed at a planned position or orientation within the model of the anatomy. After placement, the voxel values corresponding to the implant location are modified to reflect the planned configuration, resulting in planned post-implantation volume data.

From this modified volume data, one or more predicted synthetic DRs are created, which may be used to simulate post-operative imaging. The synthetic DRs can then be presented for visual comparison with intra-operative radiographs, such as those obtained from fluoroscopy, to evaluate the accuracy of implant placement relative to the surgical plan.

Volume data may refer to pre-operative data representing at least a portion of the patient's anatomy. The volume data may comprise a plurality of volume elements (voxels), each having an assigned value that may be associated with the radiographic density of the corresponding anatomical structure. In some embodiments, the volume data may be obtained through Computed Tomography (CT) scanning, predicted CT data, or derived from stereoradiographic imaging, such as EOS® biplanar X-ray imaging. A voxel may refer to a volume element within the volume data. Each voxel may contain a value, such as a CT number, that reflects the radiographic density at that spatial location. In certain implementations, voxels may be displayed in grayscale according to their density values and may be manipulated computationally to reflect changes in anatomical structure or implant placement.

The three-dimensional (3D) model of the patient's anatomy may be generated from the volume data and may comprise surface or solid representations of anatomical structures such as the pelvis or femur. In some embodiments, the 3D model may be subdivided into sub-volumes, for example, for the pelvis and the femur on the operative side.

Placing a 3D model of an implant may involve selecting a digital representation of a prosthetic component and positioning it at a planned location and orientation within the 3D model of the patient's anatomy. The implant model may include components such as a cup, liner, stem, or femoral head. The placement may be based on clinical planning parameters such as inclination, anteversion, or leg length.

The planned position or orientation may refer to the intended spatial configuration of the implant within the patient's anatomy, as defined during the pre-operative planning phase. This may include translational and/or rotational parameters relative to anatomical coordinate systems.

Translation into planned post-implantation volume data may involve modifying the voxel values of the original volume data to reflect the presence of the implant at its planned location. The values of voxels that correspond to the implant model may be changed to one or more new values, for example, representing the radiographic density of the implant material or another selected value for improved visualization.

A predicted synthetic digital radiograph (DR) may refer to a simulated radiographic image generated from the planned post-implantation volume data. Generating such DRs may involve specifying a virtual x-ray source and detector and calculating the expected radiographic projection based on voxel values. The resulting image may show the implant in its planned position or orientation as it would appear in a radiograph.

Presenting the predicted synthetic DR may involve displaying the image on a screen or other visual interface. In some embodiments, this presentation may support intra-operative comparison with actual radiographic images obtained during the procedure, for example, from a C-arm fluoroscopy system. In certain implementations, the DR may also be superimposed onto live or recorded intra-operative imaging using a mixed-reality head-mounted device (MR-HMD) or other computing platform.

FIG. 2 is a schematic illustration of an example data capture system 200 in accordance with one or more embodiments. The system 200 may include an imaging apparatus 202, such as Computed Tomography (CT) scanner, for generating patient-specific, pre-operative volume data 204. The volume data may be in the form of files or objects. The data capture system 200 may further include or be in communication with a surgical planning system 300. Specifically, data, such as the volume data 204, may be obtained for a patient who is to undergo a surgical procedure. The volume data 204 may be obtained of that portion of the patient's anatomy on which the surgery is to be performed. For example, a patient may be diagnosed with hip joint failure, and may require total hip arthroplasty (THA) surgery. In this case, one or more low cost, low dose CT scans of the patient's hip may be taken.

The CT scanner 202 may generate a three-dimensional (3D) data set, e.g., an array, of volume elements, i.e., voxels, each having a radiographic density value. The CT scanner 202 creates a 3D data set by scanning the patient with a series of narrow X-ray beams from different directions. The series of X-ray beams typically moves through an arc shape. Each measurement taken during the CT scan records the attenuation of each individual x-ray beam as it passes through the patient. The CT scanner 202 processes the data set to derive an attenuation value, e.g., a CT number, for each voxel. The determined value is then encoded in the respective voxel. In some cases, the voxels of an image constructed from CT data may be displayed in shades of gray where white represents the highest X-ray absorption and black corresponds to the lowest X-ray absorption.

FIG. 3 is a schematic illustration of an example three-dimensional voxel array 300 in accordance with one or more embodiments. The voxels of the array 300 have various radiographic density values as represented by the different shades of gray. The three-dimensional voxel array 300 can be manipulated and inspected in many different ways, such as along anatomical planes, e.g., axial, coronal, and sagittal.

It should be understood that patient-specific volume data may be obtained in other ways besides or in addition to CT scans. For example, one or more of Magnetic Resonance Imaging (MRI), conventional radiographs (X-rays), bi-planar or multiplanar simultaneous radiographs, or ultrasonic images, may be taken of the patient. The one or more digital images (CT, magnetic, radiographic, ultrasonic, etc.) may provide three-dimensional (3D) volume data regarding the surface and/or structure of the portion of the patient's anatomy that was scanned, e.g., the patient's hip. The volume data may be related to radiodensity in the case of CT scan data or may relate to some other density or value depending on the type of digital image used as a starting point.

The 3D volume data may be generated in other ways. For example, the 3D volume data for a patient's hip may be predicted or derived from a single image, a statistical model, one or more measurements taken of the patient's hip, etc. In certain embodiments, pre-operative volume data can include predicted computed tomography (CT) data that may be derived, for example, from stereoradiographic imaging such as obtained using EOS® biplanar X-ray imaging (ATEC Spine, Calsbad, CA) using known 3D modeling translation techniques available for example from EOS® imaging. See also, TomoRay: Generating Synthetic Computed Tomography of the Spine From Biplanar Radiographs; Zanier, et al. Neurospine 2024; 21 (1): 68-75 2024; Daniel De Wilde, et al., 2025, Strategies for generating synthetic computed tomography-like imaging from radiographs: A scoping review, Medical Image Analysis, Volume 101, 103454, ISSN 1361-8415. Regardless of source, the obtained volume data may be transferred to the surgical planning system 300, as indicated by arrow 208.

FIG. 4 is a schematic illustration of an example of the surgical planning system 400 in accordance with one or more embodiments. The surgical planning system 400 may include a user interface (UI) engine 402, a modeling tool 404, a planning tool 406, a predictive synthetic digital radiograph (DR) generator 408, and a data store 410. The surgical planning system 400 may include or have access to a display 412. The surgical planning system 400 may receive the patient-specific volume data 204. The surgical planning system 400 may create one or more surgical plans, such as plan 414, for the surgical procedure, e.g., THA. The surgical planning system 400 also may generate one or more synthetic DRs 416. In some embodiments, the system 400 may also include a hologram generator 418 and may generate one or more holograms 420 or files from which the one or more holograms 420 may be generated.

Suitable tools for generating 2D and/or 3D models of anatomical structures from volume or shape data include the OsiriX image processing software from Pixmeo SARL of Bernex Switzerland, the TraumaCad pre-operative planning system, the HipInsight system from Surgical Planning Associates of Boston, Massachusetts, and the MAKOplasty Total Hip Application pre-operative and intra-operative planning system. Nonetheless, those skilled in the art will understand that other image processing software may be used.

One or more of the patient data 204, the surgical plan 414, the synthetic DRs 416, and the holograms 420 may be implemented through one or more data structures, such as files, objects, etc., stored in the electronic memory of a data processing device.

One or more components of the surgical planning system 400 may be or may include software modules or libraries containing program instructions pertaining to the methods described herein, that may be stored on non-transitory computer readable media, and executed by one or more processors of a data processing device. In some embodiments, one or more components of the surgical planning system 400 may each comprise registers and combinational logic configured and arranged to produce sequential logic circuits. In other embodiments, various combinations of software and hardware, including firmware, may be utilized to implement the present disclosure.

As noted, the surgical planner may select one or more prosthetic components, e.g., implants, to be used in a surgical procedure, such as a cup component and/or a femoral stem component and plan their placement in the patient's body. The plan for the cup component in addition to the make, model, and size of cup component may include a planned position, including a depth and/or an orientation within the acetabulum. The plan may also include the shape of the cup bed at the patient's acetabulum to receive the cup component. For the femoral stem component, the plan may identify the make, model, and size of femoral stem and may specify the position of the femoral stem component within the femur and/or its orientation relative to the femoral coordinate system.

In some embodiments, the plan may incorporate 3D models of one or more tools, such as a registration and tracking device, an acetabular reamer, and a cup impactor, among others.

FIG. 5 is an illustration of an example planning window 500 generated by the surgical planning system 400 and presented on the display 414 in accordance with one or more embodiments. The planning window 500, which may be created by the UI engine 402, includes a model pane 502 presenting a 3D model of the patient's hip 504 and a planning pane 506. The hip model 404 may be a 3D surface model generated by the modeling tool 504 from the volume data 204, e.g., the CT scan data. In other embodiments, the hip model 504 may be a surface model. User controls 508a-c, such as rotate, translate, and zoom in/out, respectively, may be provided for controlling how the hip model 504 appears in the model pane 502. The hip model 504 may include the pelvis 510 and the operative femur 512, e.g., the right femur. In other embodiments, modeling tool 404 may generate and operate on 3D solid models and/or a combination of surface and solid models.

The hip model 504 may present the patient's hip as captured during the CT scan, e.g., with the right native hip joint. The modeling tool 404 may be configured to create sub-volumes from the original volume data 204. More specifically, the modeling tool 404 may create a pelvis sub-model that includes the patient's pelvis and a femur sub-model that only includes the operative side femur, e.g., the right femur. In some embodiments, the modeling tool 400 may create a sub-volume by changing the radiographic density of the voxels that are not part of the sub-volume to a value for soft tissue such that the voxels will not appear on a synthetic DR. In other embodiments, the modeling tool 404 may create the sub-models by removing the voxels from the original volume data 204 that correspond to structures or elements not included in the particular sub-model. For example, the modeling tool 404 may perform a segmentation step in which 3D surface models of the pelvis and femur are created. In some embodiments, the modeling tool 404 may perform segmentation by applying a threshold that can separate bone from soft tissue. If the joint is arthritic, however, and the femur and pelvis are in contact with each other then, in additional to applying a threshold, the segmentation may be fine-tuned or completed manually. For example, as an additional segmentation step, the planner may use an edit tool in 2D or 3D, such as a brush tool configured to perform subtraction brush to “minus” out the femoral head so that then when the modeling tool 404 creates the pelvis model, it will not include the femur. Then in the second step, now that the pelvis model has been created, the modeling tool 404 can automatically “minus out” the pelvis to create a model of the femur that doesn't jump over to the pelvis. In this way, the modeling tool 404 can generate separate models of both the operative femur and the pelvis. The modeling tool 404 and/or the predictive synthetic DR generator 408 can the use the models as masks, e.g., to edit the voxels within the femoral model for example. To model the pelvis and the cup component, the modeling tool 404 may use the 3D surface model of the femur as a mask. The predictive synthetic DR generator 408 may take all of the voxels that exist inside of the femur model and change their values, e.g., to new radiographic density values, such as soft tissue, air, etc. In addition, since the modeling tool 404 knows where the cup component will be (position and/or orientation), the modeling tool 404 may identify the volume of bone that is overlapping the volume of the cup component and remove that volume of bone so it is not shown in the planning images because it would have been removed by the surgery. That is, the modeling tool 404 can determine where two objects (the acetabular bone and the cup) are overlapping and determine which object to keep and which to remove. In this case, the modeling tool 404 may remove the bone to show the effect of what an intraoperative x-ray image would look like after reaming the patient's acetabulum. The predictive synthetic DR generator 408, moreover, can create a predictive synthetic DR that shows only the pelvis with the femur removed and with the reamed acetabular bone removed.

On the femur side, the patient's femur may have been in a distorted position when the volume, e.g., CT, data was obtained, such that if a predictive synthetic DR perpendicular to the coronal plane of the femur is created, the patient's other femur may also be in predicted synthetic DR. When creating predictive synthetic DRs of the planned reconstruction of the femur, the predictive synthetic DR generator 408 may remove the pelvis volume and the contralateral femur volume. For the operative femur, the predictive synthetic DR generator 408 may define another sub-volume that includes the femoral head and neck portion to be removed during the surgery, e.g., by a neck cut with a saw. It may be removed using mask as described. The position and/or orientation of the selected femoral component with the femoral head component may be planned. The predictive synthetic DR generator 408 may then generate one or more predictive synthetic DRs relative the femoral coordinate system. In some embodiments, the predictive synthetic DR generator 408 may “stitch” a predictive synthetic DRs of the planned femur onto a predictive synthetic DR of the reconstructed pelvis with the two sets of predictive synthetic DRs joined at the respective centers of rotation of the acetabular insert and the femoral head, thereby completing the predictive process. Further, if the surgeon changes the plan, the surgical planning system 400 could re-mask the volume/sub-volume data and generate new predictive synthetic DRs.

The modeling tool 404 also may create 3D models for the different sub-models, e.g., pelvis, femur, etc. The planning pane 506 may include an area 514 through which the planner may select, e.g., using checkbox elements, which sub-models to display on the model pane 502.

The planning pane 506 also may include sub-panes containing user interface elements for planning the surgery. For example, the planning pane 506 may include a ‘Cup Plan’ sub-pane 516 for planning the cup component and a ‘Stem Plan’ sub-pane 518 for planning the femoral stem.

FIG. 16 is a schematic illustration of an example three-dimensional voxel array 1600 in accordance with one or more embodiments. As indicated, the modeling tool 404 may identify particular voxels of the array 1600 as part of a pelvis sub-volume 1602 and other voxels as part of a femur sub-volume 1604. The pelvis sub-volume may include voxels 1606 and 1608

FIG. 6 is an illustration of another example planning window 600 in accordance with one or more embodiments. Here, the operative, e.g., right, femur is removed and models 602 and 604 of the selected cup component and liner are displayed at the patient's acetabulum. In addition, an image 606 representing the Anterior Pelvic (AP) Plane is shown. In some embodiments, the planner may plan the location, e.g., position and orientation, of the cup component 602 in the acetabulum relative to the AP Plane 606. For example, the planning pane 406 may include elements 608 and 610 for setting the operative anteversion (OA) and operative inclination (OI) of the cup component. The elements 608 and 610 may include sliders, numeric data entry boxes, and plus/minus controls.

After planning the position and/or orientation of the cup component 602, a determination may be made of acetabular osteophytes of the pelvis model 510 that are sticking out, e.g., potentially in the way. A large sphere masking edit tool (see FIG. 18) may be placed basically at the center of rotation of the cup component and used to “cut” the osteophytes from the rest of the pelvis model. In some cases, the modeling tool 404 may define the osteophytes as separate objects. The radius of the sphere masking edit tool may be adjustable.

With osteophytes removed, the surgical planning system 400 can simulate range of motion of the replacement hip and predict bone-bone, implant-bone, and implant-implant impingement. An edit tool in the form of a sphere that is larger than the acetabulum can be provided in the window 502 and positioned within the acetabulum so that the sphere covers the acetabular osteophytes. The modeling tool 404 may remove acetabular bone covered by the edit tool from the pelvis model 510. Alternatively, the density values for the voxels of the volume data that are overlapped by the tool may be changed from their original values to new density values that essentially remove them.

FIG. 18 is a schematic illustration of a planning window 1800 in accordance with one or more embodiments. The model plane 502 may include a edit tool 1802 in the form of a partial sphere which may be positioned at the acetabulum to cover the acetabulum osteophytes.

In some embodiments, the patient's pelvis or femur may be registered and tracked during the surgical procedure. For example, a physical device may be docked to the patient's pelvis to register the pelvis. The device may also be tracked to maintain registration of the pelvis during the procedure. In some embodiments, the planning phase may include planning the device used to register and track the patient's pelvis.

FIG. 7 is an illustration of another example planning window 700 in accordance with one or more embodiments. Here, a 3D model 702 of a registration and tracking device may be docked to the model of the pelvis 502. The model of the device 702 may include a tracker 704 that can be detected by a MR HMD. A code on the tracker may expose a spatial 3D coordinate system. The planning tool may construct a translation matrix that translates positions and/or orientations from the AP Plane coordinate system to the spatial coordinate systems of the tracker 704. If utilized, the planner may determine one or more adjustments to the registration and tracking device in order for the physical device to dock to the patient's pelvis as planned.

The surgical planner may plan the position, shape and orientation of the cup bed to receive the cup component, e.g., to achieve the one or more goals of the surgery. The cup bed refers to the ideal surgically created bone surface to receive the cup component in the planned position and/or orientation.

In some embodiments, computer-generated, three-dimensional (3D) models, such as Computer Aided Design (CAD) models, of one or more surgical tools may be stored in the data store 410. 3D surface models of the surgical tools may be generated from these models and also stored in the data store 410. In some embodiments, only the 3D surface models may be included in the data store 410. In some embodiments, 3D surface models of one, a handful or some other small number of standard surgical tools, such as a standard acetabular reamer with a standard cutting basket and a standard acetabular cup impactor may be included in the data store 410. Holograms that include a reamer or cup impactor may be based on these surface models of a standard reamer or cup impactor.

However, in other embodiments, 3D models for actual reamers and/or cup impactors including entire product families from one or more manufacturers, e.g., Stryker Corp. of Kalamazoo, MI, Greatbatch, Inc. (now Integer Holdings Corp.) of Plano, TX, Ortho Solutions UK Ltd. of Essex, UK, Zimmer Biomet Holdings, Inc. of Warsaw, IN, Depuy Synthes of Raynham, MA, etc., may be included in the data store 410. Furthermore, 3D models for different sizes of cutting baskets and different sizes of acetabular cups may be included in the data store 410. During the surgical planning phase, 3D models corresponding to the particular reamer and the particular cup impactor that the surgeon will be using in the surgery may be selected from the data store 410 and used in creating the surgical plan. 3D models for cup impactors and cup components may even include spatial assembly information for how each of the planned cup assembles onto the cup impactor, e.g., due to thread depth and shell thickness). In this way, synthetic DRs representing the particular surgical tools that the surgeon is using may be generated and presented.

In some embodiments, the surgical planner may determine the location of an acetabular reamer at the 3D model of the pelvis, e.g., relative to the AP Plane coordinate system, to prepare the cup bed as planned. For example, the acetabular reamer may have a handle defining a longitudinal axis. The surgical planner may position a 3D model of the acetabular reamer so that the cutting basket of the reamer is positioned in the acetabulum to prepare the cup bed as planned.

FIG. 8 is an illustration of another example planning window 800 in accordance with one or more embodiments. In addition to the pelvis model 510, the model pane 502 also includes a model of a reamer tool 802. The planner may position and/or orient the reamer tool 802 at the final position and/or orientation for preparing the patient's acetabulum to receive the cup component.

The surgical planner also may determine the location of a cup impactor at the 3D model of the pelvis, e.g., relative to the AP Plane coordinate system, to implant the cup component in the cup bed as planned. For example, the cup impactor may have a handle defining a longitudinal axis. The surgical planner may position a 3D model of the cup impactor so that the longitudinal axis defined by the handle positions the cup component at the end of the cup impactor in the cup bed as planned.

FIG. 9 is an illustration of another example planning window 900 in accordance with one or more embodiments. In addition to the pelvis model 510, the model pane 502 also includes a model of a cup impactor tool 902. The planner may position and/or orient the cup impactor tool 902 at the final position and/or orientation for implanting the cup component in the patient's acetabulum at the planned position and/or orientation.

As noted, in addition to planning the cup component, the planner may also plan the femoral stem component.

FIG. 10 is an illustration of another example planning window 1000 in accordance with one or more embodiments. In addition to the pelvis model 510, the cup component 602, and the liner 604, the model pane 502 also includes a model of the stem component 1002 as planned.

The predictive synthetic DR generator 408 may generate the one or more synthetic DRs 416 as the surgical plan 414 is being created or once it is completed. The predictive synthetic DR generator 408 may create the one or more synthetic DRs 416 based on the predicted volume data generated during the creation of the surgical plan 414. For example, the predictive synthetic DR generator 408 may generate synthetic DRs from the 3D models created for the surgical plan.

Referring to FIG. 7, which shows the cup component and liner in the planned position and/or orientation in the acetabulum, the predictive synthetic DR generator 408 may create a synthetic DR from predicted volume data generated for this model. The registration and tracking device may not be included in the predicted volume data from which the synthetic DR is created.

FIG. 11 is an illustration of an example planning window 1100 presenting a synthetic DR 1102 as generated in accordance with one or more embodiments. The synthetic DR 1102 shows an x-ray image of the pelvis 1104 and an x-ray image of the cup component 1106 as implanted at the acetabulum in the planned position and/or orientation. In some embodiments, the predictive synthetic DR generator 408 may generate the synthetic DR 1102 as follows. The predictive synthetic DR generator 408 may remove the voxels for the operative femur. For example, the predictive synthetic DR generator 408 may only utilize the sub-volume created for the pelvis. The predictive synthetic DR generator 408 may identify the voxels from the original volume data, e.g., the sub-volume for the pelvis, that correspond to the acetabular cup component as located in the planned position and/or orientation in the acetabulum. The predictive synthetic DR generator 408 may then change the values of these identified voxels from their original values to values corresponding to the radiographic density of the material from which the cup component is fabricated, e.g., titanium.

Another exemplary planning window 2500 is shown in FIG. 25 presenting a synthetic DR 2502. Similarly to FIG. 11, the synthetic DR 2502 shows a synthetically generated x-ray image of the pelvis 1104 and a synthetically generated x-ray image of the cup component 1106 as implanted at the acetabulum in the planned position and/or orientation. For side-by-side comparison, an operating room (OR) team can put a display showing the predictive synthetic DRs right next to the fluoroscopic display and an x-ray angle of the predicted radiograph that most closely matches the actual fluoroscopic image taken can be selected so that the OR team can visually compare the two to verify that the actual positioning/alignment of tools, components, and anatomy matches the planned, optimum positioning/alignment. Pre-selected synthetic DRs with various elements, such as anatomic elements, implants, tools, etc., synthetically removed or added can be selected by the OR team via a drop down menu 2501 as shown in FIG. 25. A user can also select a desired angle for the predictive synthetic DR to be shown using the buttons 2503.

FIG. 17 is a schematic illustration of an example three-dimensional voxel array 1700 accordance with one or more embodiments. The array 1700 may represent predicted volume data for the pelvis created by the predictive synthetic DR generator 408. For example, the predictive synthetic DR generator 408 may identify the voxels from the original pelvis sub-volume 1602 (FIG. 16) that now correspond to a prosthetic component as placed in a planned position and/or orientation relative to the patient's pelvis, such as the cup component. The predictive synthetic DR generator 408 may change the values of these voxels from their original values to new values that represent the radiographic density of the cup component. For example, suppose the predictive synthetic DR generator 408 determines that voxels 1606 and 1608, which were originally shaded light gray, from the original pelvis volume 1602 correspond to the cup component. The predictive synthetic DR generator 408 may change the density values associated with these voxels in the predicted volume data 1700. For example, in the predicted volume 1700, the shading of voxels 1702 and 1704 (which correspond to voxels 1606 and 1608 from the original volume data) is changed from light gray to white.

As noted, the predictive synthetic DR generator 408 may change the voxels for the cup component to a density value for the cup material, e.g., titanium. In other embodiments, the predictive synthetic DR generator 408 may use other density values, e.g., values that do not match the cup material. For example, to improve the cup image on the synthetic DRs, the predictive synthetic DR generator 408 may use a different density value than the density value for titanium. In some embodiments, the selected density value may not match any material. In this way, the predictive synthetic DRs can provide more detail than what may be shown in an actual intraoperative x-ray image. For example, often times, a radiograph or fluoroscopic image of the acetabulum component may only show one rim of the acetabulum, usually the posterior rim on an AP or PA view. This is due to the density of titanium, especially when the acetabular cup component has a relatively larger thickness. In such situations, if the density for the voxels representing the titanium component are designated to be lower than the actual density of titanium, the amount of information and detail on the predictive synthetic DR can be superior to that of an actual x-ray image.

This process of identifying voxels from the original volume data that now correspond to prosthetic components and changing the original density value to a new value, which may correspond to the material of the prosthetic component (or may be some other value) may be performed for the other prosthetic components, such as liner and the stem component. The voxels for the liner, which may be formed from plastic, may be changed to a value corresponding to the type of plastic used to create the liner.

The predictive synthetic DR generator 408 may determine a virtual source point relative to the predicted volume data and a virtual image plane relative to the predicted volume data opposite the source point. The predictive synthetic DR generator 408 may determine the projection of x-ray beams from the virtual source point to the virtual image plane. In some embodiments, one or more of these settings may be entered by the planning, e.g., through a UI. In other embodiments, default values may be used. Exemplary default values may set the center of the x-ray beam on the hip center and the distance between the x-ray source and image plane distance to a value for a typical fluoroscopy machine setup in an operating room. The predictive synthetic DR generator 408 may then calculate the radiographic density that would reach the virtual image plane to construct a synthetic DR. The predictive synthetic DR generator 408 may generate either or both Anterior-Posterior (AP) synthetic DRs and Posterior-Anterior (PA) synthetic DRs. In some embodiments, a toggle switch or other user element may be provided to select AP or PA synthetic DRs.

A C-arm fluoroscopy machine may be rotated about two orthogonal axes, which may be referred to as rotation and angulation. The predictive synthetic DR generator 408 may be configured to generate synthetic DRs that correspond to different rotation and angulation angles. In some embodiments, the predictive synthetic DR generator 408 may generate a first synthetic DR that may correspond to the functional plane of the CT gantry. This synthetic DR may be designated as 0,0 rotation and angulation. The predictive synthetic DR generator 408 may then generate additional synthetic DRs that change the rotation and angulation in increments of five degrees, such as:
0,5 0,10 0,15 0,20
5,0 10,0 15,0 20,0
5,5 5,10 5,15
10,5 15,5, 20,5
etc.

The predictive synthetic DR generator 408 may generate additional and/or other synthetic DRs. For example, with reference to FIG. 8, the predictive synthetic DR generator 408 may generate one or more synthetic DRs that show the reamer tool in the planned position and/or orientation to prepare the acetabular cup bed to receive the cup component. Alternatively, the MR HMD could use a 2D/3D matching algorithm to determine the characteristics of the intra-operatively acquired x-ray image and then direct the predictive synthetic DR generator 408 to generate a predictive synthetic DR of the same spatial characteristics for qualitative or quantitative or automated analysis. In this case, pre-generating predictive synthetic DRs, e.g., in 5 degree or 2 degree increments, may be avoided.

An example synthetic DR 2301 without any predictive CT volume edits is shown in FIG. 23. As seen, all elements, including the pelvis 1104 and the femur 2303 are seen as they would be expected to present in a digital radiograph obtained at the specified angle pre-surgery. FIG. 24 shows a synthetic DR 2401 for the same patient generated from the same position and angle, but with the femur removed by using the 3D volume of the femur 2303 as an edit tool and changing all of the voxels within that volume to have, for example, values for soft tissue density. In other embodiments, other values besides values for soft tissue may be assigned to the voxels. In other embodiments, combinations of different values may be assigned to the voxels, such as values for soft tissue and other values.

Just as voxel values may be edited as shown in FIG. 24 to appear to remove elements such as the femur 2303, voxel values can be edited to include additional elements such as an acetabular cup component 1106 at the optimal or planned position and orientation as shown in FIG. 11. The predictive radiograph in FIG. 11 is of the pelvis side of the planned surgery with the femur 2303 removed and the cup 1106 and cup liner components inserted. If the cup liner had a metal ring locking mechanism, were made of CoCr alloy, or were made of ceramic or any other material, the predictive radiograph could be generated with voxel values for those parameters as well.

FIG. 12 is an illustration of an example synthetic DR 1200 in accordance with one or more embodiments. The synthetic DR 1200 includes the pelvis 1104 and an x-ray image of a reamer tool 1202. Knowing the position and orientation of the planned cup allows the acetabular reamer tool 1202 to be added into the predictive radiographs as well. So instead of having the cup and liner in the predictive radiograph, the femur is removed and then the reamer handle and reamer basket are added. FIG. 12 shows a synthetic DR with an “offset” reamer handle that is often used during the surgical procedure.

With reference to FIG. 9, the predictive synthetic DR generator 408 may generate one or more synthetic DRs that show the cup impactor tool in the planned position and/or orientation to implant the cup component as planned.

FIG. 13 is an illustration of an example synthetic DR 1300 in accordance with one or more embodiments. The synthetic DR 1200 includes the pelvis 1104 and a predicted x-ray image of a cup impactor tool 1302. Knowing the position and orientation of the planned cup allows for the addition of predicted voxel data for the cup impactor itself into the predictive radiographs. So, as before, the femur is removed and the volumes for the cup and cup impactor are edited in the raw CT volume before image generation, resulting in the predictive synthetic DR 1300 shown in FIG. 13.

It should be understood that the synthetic DRs of FIGS. 11-13 are meant for illustrative purposes and that the predictive synthetic DR generator 408 may create other and/or additional synthetic DRs.

The planning tool 406 can also create a femoral plan that includes the selected femoral stem and its position and/or orientation at the patient's femur. In some embodiments, the planning tool 406 may determine a cut plane for resecting the femur using the selected femoral stem positioned at the planned position and/or orientation at the femur.

FIG. 19 is a schematic illustration of a front view of an example femoral component 1900 in accordance with one or more embodiments. The modeling tool 404 may define a point 1902 on the medial side of the femoral component 1900 the base of a collar if included on the selected femoral component or at the upper edge of the porous coating applied to femoral component if no collar is present. The planning tool 406 may define a plane 1904 that includes the medial point 1902 and extends along the base of the collar if present or along the upper edge of the porous coating. The planning tool 406 may transfer this cut plane to the femur model.

FIG. 20 is a schematic illustration of a side view of an example femur model 2000 in accordance with one or more embodiments. The planning tool 406 may include a cut plane 2002 on the femur model based on the plane 1904 defined for the femoral component 1900.

The planning tool may plan a counter-cut at the femur, e.g., to preserve the greater trochanter. The planning tool 406 may plan the counter-cut to be parallel with the long axis of the femoral component 1900 and perpendicular to the medial-lateral axis of the femoral component 1900. The position of the medial-lateral axis may be adjusted, for example due to patent anatomy and/or attributes of the femoral component.

FIG. 21 is a schematic illustration of another front view of the femur model 2000 in accordance with one or more embodiments. A counter-cut plane 2102 as planned is shown. The counter-cut plane 2102 is adjustable as shown by arrow 2104.

FIG. 22 is a schematic illustration of a top view of the femur model 2000 and a top view of the femoral component 1900 in accordance with one or more embodiments. The counter-cut plane 2102 and the arrow 2104 are shown at the femur model. A corresponding counter-cut plane 2202 is also shown at the femoral component model 1900.

With the femur planned, the predictive synthetic DR generator 408 can create additional sub-volumes for the femur. For example, the synthetic DR generator 408 may create one partial femur sub-volume that includes the voxels below the cut plane 2002 and the counter-cut plane 2102. The predictive synthetic DR generator 408 may create another partial femur sub-volume that includes the voxels above the cut plane 2002 and the counter-cut plane 2102. The predictive synthetic DR generator 408 may include the partial femur sub-volume below when creating synthetic DRs for the hip reduced with the femoral component and the acetabular component. For example, a predictive synthetic DR may just have the femur with the head and neck removed and the femoral stem and head at the planned position and/or orientation. The “0,0” predictive synthetic DR may be orthogonal to the coronal plane of the femur and centered on the femoral head center. The predictive synthetic DR generator 408 may mask out the pelvis and the contralateral femur which could otherwise be in the background if the leg position was contorted during the CT scan. It should be understood that in addition to generating predictive synthetic DRs of the femur and femoral component, predictive synthetic DRs may be generated of planned reamers, femoral preparation broaches with or without neck trials or broach handles, for example.

In many cases, a patient's femur may be mal-positioned when the CT scan of the pelvis is taken. For example, the femur may be externally rotated. It also or additionally may be significantly rotated in abduction and/or flexion. FIG. 26 shows a CT scan 2601 of a severely malpositioned femur 2603 in which the patient is lying in the CT scanner with their femur flexed severely, for example due to a neuromuscuar imbalance and secondary arthritis. In practicality, almost all femurs are malpositioned to some degree in pre-operative CT scans compared an ideal positioning a surgeon might wish to see before predictive synthetic DRs are generated. Therefore, in certain embodiments, it may be desirable to take a whole femur sub-volume and move it in a new position within the voxel data before generating predictive synthetic DRs according to the methods described herein. This repositioning may be used both to generate holograms with the femur and pelvis in new and relevant positions relative to each other and also for generation of predictive synthetic DRs which have both the pelvis and femur in them in the anticipated/desired orientation and relationship post-surgery.

In certain embodiments, femur scans and predictive synthetic DRs may be prepared using a femoral coordinate system while pelvis scans and predictive synthetic DRs may be prepared in a pelvic coordinate system. The two components (femur and pelvic) can then be combined. This combination allows for the generation of synthetic DRs of the femur with implants using the femur sub volume relative to the femoral coordinate system or the option to reposition the femoral volume around into position relative to the pelvic coordinate system before generating synthetic DRs.

Repositioning can be performed at various stages of synthetic DR generation including before modification of the 3D voxel data with planned implants. As noted above, repositioning can be performed by the predictive synthetic DR generator 408. In some embodiments, the two-dimensional synthetic DR of the femur may be connected to the pelvis at the planned cup center-femoral head center or the femur CT volume may be moved around so that the volume merges with the Pelvis CT volume (relative to the AP Plane (APP) coordinate system (COS)), again at the planned cup center/femur head center.

When positioning of the femur in the CT scan is not ideal, the femoral voxel volume may be repositioned according to a variety of desired relationships between the femur and one or more references. For example, the following options may be used to inform repositioning of the femur:
The center of the prosthetic femoral head can be repositioned to be coincident with the center of a planned acetabular component.
Coronal alignment of the femoral volume can be positioned so that a long axis defined by the prosthetic head center to the center of the knee is perpendicular to the medial lateral axis of the pelvis.
Rotationally, the femoral volume can be positioned so that the axis between the medial and lateral posterior condyles is parallel with the medial-lateral (m-l) axis of the pelvis.
The sagittal alignment of the femoral volume can be positioned so that the long axis defined by the prosthetic head center to the center of the knee is placed according to the pelvic tilt (e.g., supine from the plan or a number entered from measurement of a standing view). For example, if the pelvic tilt is plus 5 degrees, the femur can be virtually flexed around the m-l pelvis axis 5 degrees relative to the APP to accomplish the desired repositioning.

In various embodiments, this repositioning can be accomplished automatically through identification of the relevant landmarks in any portion of the anatomy (e.g., femoral head center, condyles, knee center, or pelvis) either through manual tagging or automated image recognition. Such automated femoral repositioning in the raw CT coordinate space in relation to the pelvis can allow the generation of predictive synthetic DRs and holograms reflecting an ideally positioned planned reconstruction or altered to more closely match the anticipated views from intra-operative fluoroscopic images.

The predictive synthetic DR generator 408 may reposition the femur sub-volume and/or a femur partial sub-volume before or after generating one or more synthetic DRs, e.g., to correct for a mal-positioned femur. In some embodiments, the predictive synthetic DR generator 408 may adjust the femur sub-volume and/or a femur partial sub-volume as described above. With the femur sub-volume repositioned, the predictive synthetic DR generator 408 may generate the one or more synthetic DRs.

The surgical plan 414, the synthetic DRs 416, and the holograms 420 may be transmitted to the surgeon performing the surgical procedure who may review them.

FIG. 14 is a schematic illustration of an operating room 1400 in accordance with one or more embodiments. Disposed in the operating room 1400 may be a patient 1402 on an operating table 1404, a C-arm fluoroscopy machine 1406, and a surgeon 1408. The surgeon 1408 may be using the anterior approach. For surgery with the patient in the lateral position, a plain x-ray machine is used rather than a C-arm fluoroscopy machine. A plain x-ray machine does not have a rigid connection between the x-ray source and the detector. To generate predictive synthetic DRs for this case, the predictive synthetic DR generator 408 may utilize default settings for the plain x-ray machine, such as source to film distance, center, position of the patient within that projection, etc. In some embodiments, the settings may be specified by the user, e.g., the planner. The C-arm fluoroscopy machine 1406 may be set up to image the patient's pelvis. The fluoroscopy machine 1406 may be configured to capture still images and/or a series of images, e.g., 25-30 per section, that are converted to video format. The fluoroscopy machine may include an x-ray source 1410 and a detector 09. The detector may be a digital capture device (as opposed to radiographic film). The C-arm fluoroscopy machine 1406 may rotate the source 1410 and the detector 1412 about two axes, such as a rotational axis indicated by arrow 1414 and an angulation axis indicated by arrow 1416.

One or more monitors, such as monitors 1418 and 1420, may also be disposed in the operating room 1400. The two monitors 1418 and 1420 may be arranged side-by-side. The surgeon 1406 may be wearing a Mixed Reality (MR) Head Mounted Device (HMD) 1414. Intra-operative images generated by the C-arm fluoroscopy machine 1406 and the synthetic DRs may be displayed on the one or more monitors. In some embodiments, the intra-operative x-ray images may be displayed on one of the monitors, e.g., monitor 1418, while the synthetic DRs may be displayed on the other monitor 1420. In other embodiments, the intra-operative x-ray images and the synthetic DRs may both be displayed on the same monitor.

In various embodiments, the DR generator 408 can generate synthetic DRs with one or more graphical affordances added to the synthetic DRs, such as an error gauge 2701, as shown in FIG. 27. In other embodiments, other information may be included in the synthetic DR by the DR generator 408 such that, when the synthetic DR is overlayed onto an intraoperative fluoroscopic image, a user can visually discern and thus quickly identify an error and its severity (e.g., an offset from the planned acetabular cup orientation or positioning). Such graphical affordances, such as gauges 2701, can, for example, show +/−5° of inclination and +/−5° of anteversion to allow a surgeon to easily identify if the cup placement is correct or, if not, to determine how many degrees the placement is off from the plan. Similar error gauges or other graphical affordances can be included for leg length and offset as well and used in the planned surgical images where a planned surgical placement is desired.

Exemplary reference marks or rulers 2801 for length and offset of the femur are shown in FIG. 28. The vertical (length) 2803 and horizontal (offset) 2805 gauges can be centered or otherwise placed with reference to any anatomical or component landmark at the intended location and orientation from the surgical plan (e.g., centered at the tip of the greater trochanter). Those marks can provide a quick reference for a user to identify and quantify any vertical or horizontal deviation of the intra-operative or post-operative implant or anatomical portion relative to the pre-operative plan by overlaying the predictive synthetic DR with the graphical affordances, such as the error gauge marking and the actual intra-operative or post-operative imaging of the patient.

The error gauges can take, for example, the form of annotations to the synthetic DR. In various embodiments, the display device, such as the HMD MR device, among others, can be operated to toggle pre-selected error gauges on or off on the displayed synthetic DR according to a user's preferences. With respect to the example shown in FIG. 27, the error gauge 2701 relates to positioning of a planned acetabular cup implant and, therefore, can be automatically sized and positioned as part of or based on the 3D model of that component used by the system to create the modified voxel values and synthetic DRs. The graphical affordance, such as the gauge, can take the form of any series of lines, dots, animations, or other symbols. As shown in FIG. 27, a central line 2703 connecting two points on the rim of the planned cup indicates correct placement and other lines can be included at varying degrees of positive or negative offset or rotation. Upon viewing the synthetic DR in an overlay with intra-operative fluoroscopic images, a user can then compare the actual positioning of the real-world implant with the planned positioning and readily observe a degree of offset or rotation based on how that real-world implant aligns with the error gauge. In addition to rotation, dots or lines can be included showing a planned depth of a portion of the implant (e.g., the uppermost surface of an acetabular cup) along with varying distances (e.g., in mm) of greater and lesser depth along the axis of implantation.

FIG. 15 is an illustration of an example intra-operative display 1500 in accordance with one or more embodiments. The display 1500 may include an intra-operative x-ray image 1502, e.g., generated by the C-arm fluoroscopy machine 1406, showing a cup component implanted in a patient's acetabulum and a synthetic DR 1504 showing the planning position and/or orientation of the cup component. The intra-operative x-ray image 1502 includes the resected femur, while the femur is not included in the synthetic DR 1504.

In some embodiments, the synthetic DR. 1504 may have a much larger field of view than the intra-operative x-ray image 1502. In some embodiments, the synthetic DR generator may apply a mark to one or more synthetic DRs to indicate the field of view of an x-ray image generated by a fluoroscopy machine. For example, the synthetic DR generator may add a circle to the synthetic DR to depict the image size of an intra-operative x-ray image from the fluoroscopy machine.

In some embodiments, one or more of the synthetic DRs may be printed on a clear, e.g., transparent, film or other medium. During the surgical procedure, the printed synthetic DR may be placed over the intra-operative x-ray image displayed on a monitor in order to compare the planned position and/or orientation of components and the physical components actual positions and/or orientations as shown on the intra-operative x-ray images. The predictive synthetic DR generator 408 may modify synthetic DRs that are intended to be printed. For example, the predictive synthetic DR generator 408 may only include the contours of bones and implants on printed synthetic DRs making it easier to overlay the printed synthetic DR onto a displayed image of an intra-operative x-ray image and to compare the two.

In some embodiments, the predictive synthetic DR generator 408 may create a sub-volume of the operative femur in which the femoral head and neck are removed based on the planned resection of the femur and/or on the position and/or orientation of the planned femoral component. The predictive synthetic DR generator 408 may also combine this femoral sub-volume with the pelvis sub-volume by locating the center of the prosthetic femoral head into the center of the acetabular cup component of the pelvis sub-volume. In some cases, the femoral sub-volume may be rotated, e.g., so that the condylar plane is parallel with the horizontal of the CT gantry. The predictive synthetic DR generator 408 may then generate one or more synthetic DRs of the entire planned reconstruction of the patient's hip based on these combined sub-volumes. The synthetic DR, which is being created pre-operatively, would mimic a post-operative x-ray image.

In some embodiments, the MR HMD 1414 may access and display the synthetic DRs generated by the predictive synthetic DR generator 408. The position of a synthetic DR as presented by the MR HMD 1414 may controlled manually or with a tracker. With manual control, the surgeon 1408 may use inputs recognized by the MR HMD 1414, such as hand gestures, voice commands, etc. With tracker control, the surgeon can pick-up a sterile tracker and the MR HMD 1414 can display a synthetic DR as anchored to the tracker. In this way, by moving the tracker around, e.g., by and, the surgeon 1408 can control where the synthetic DR is displayed by the MR HMD 1414.

In some embodiments, the MR HMD 1414 may display thumbnail images of the available synthetic DRs. The surgeon 1408 can choose a particular synthetic DR from the thumbnail images and the MR HMD 1414 can display the selected synthetic DR. The surgeon can control the selected synthetic DR, e.g., either manually or with a tracker. In various embodiments, the visuals displayed on the MR HMD may be simultaneously shown, via wired or wireless connection, on an external display such as a television or monitor for other individuals in the OR to see. In some embodiments, synthetic DRs may be superimposed on a fluoroscopic display, either manually with a physical transparent overlay, or digitally on the display itself.

In some embodiments, the predictive synthetic DR generator 408 may be utilized during the surgical procedure to generate additional, e.g., new synthetic DRs. The predictive synthetic DR generator 408, which may be loaded onto a laptop or other data processing machine, may be accessible to the surgeon 1408 in the OR 1400. The MR HMD 1414 may include image processing software that can analyze an intra-operative x-ray image, e.g., generated by the C-arm fluoroscopy machine 1406 and presented on the monitor 1418 and determine the parameters for generating an equivalent synthetic DR from the predicted volume data. The predictive synthetic DR generator 408 may utilize the parameters determined by the MR HMD 1414 and generate a corresponding synthetic DR. The synthetic DR may be presented on the monitor 1420, e.g., for comparison with the intra-operative x-ray image. The MR HMD may analyze an intraoperative x-ray image and determine how the image (e.g., angle, center, distances etc.) was generated. Knowing the position of the pelvis on that image, the MR HMD may calculate the position of the cup component on that image. In some embodiments, the predictive synthetic DR generator 408 may (but need not) generate a predictive synthetic DR. The predictive synthetic DR may be generated to match the characteristics of the intraoperative x-ray image, including the exact changes in position and orientation compared to the native hip and compared to the original plan for the reconstruction. In some embodiments, the MR HMD (or the planning system 400) may use the contours of the bone and the 2D/3D matching algorithm to “snap” the predictive synthetic DR to be perfectly superimposed onto the intraoperatively acquired x-ray image automatically (e.g., matching the scale, position, angle, etc.). In various embodiments, systems and methods of the invention may scale, rotate, pan, anchor, and/or pin predictive synthetic DRs to preset standards, operator preferences, or to match fluoroscopic images from the OR. The MR HMD (or the planning system 400) may also determine and present the error characteristics between what was planned and what was achieved during the surgical procedure. In certain embodiments, the comparison between intraoperative fluoroscopic images and predictive synthetic DRs may optionally be facilitated using conventional image analysis or alignment techniques.

The foregoing description of embodiments is intended to provide illustration and description, but is not intended to be exhaustive or to limit the disclosure to the precise form disclosed. Modifications and variations are possible in light of the above teachings or may be acquired from a practice of the disclosure. For example, while a series of acts has been described above, the order of the acts may be modified in other implementations. In addition, the acts, operations, and steps may be performed by additional or other modules or entities, which may be combined or separated to form other modules or entities. Further, non-dependent acts may be performed in parallel. Also, the term “user”, as used herein, is intended to be broadly interpreted to include, for example, a computer or data processing system, such as a computer running a chatbot or generative Artificial Intelligence (AI) system, or a human user of a computer or data processing system, unless otherwise stated.

Further, certain embodiments of the disclosure may be implemented as logic that performs one or more functions. This logic may be hardware-based, software-based, or a combination of hardware-based and software-based. Some or all of the logic may be stored in one or more tangible non-transitory computer-readable storage media and may include computer-executable instructions that may be executed by a computer or data processing system. The computer-executable instructions may include instructions that implement one or more embodiments of the disclosure. The tangible non-transitory computer-readable storage media may be volatile or non-volatile and may include, for example, flash memories, dynamic memories, removable disks, and non-removable disks.

No element, act, or instruction used herein should be construed as critical or essential to the disclosure unless explicitly described as such. Also, as used herein, the article “a” is intended to include one or more items. Where only one item is intended, the term “one” or similar language is used. Further, the phrase “based on” is intended to mean “based, at least in part, on” unless explicitly stated otherwise.

The foregoing description has been directed to specific embodiments of the present disclosure. It will be apparent, however, that other variations and modifications may be made to the described embodiments, with the attainment of some or all of their advantages. For example, the compiler 132 and the code generation system 200 may be combined into a single entity. Therefore, it is the object of the appended claims to cover all such variations and modifications as come within the true spirit and scope of the disclosure.

What is claimed is:

1. A computer-implemented method comprising:
receiving pre-operative volume data for at least a portion of a patient's anatomy, the volume data including a plurality of volume elements (voxels) having assigned values associated with radiographic density of the portion of the patient's anatomy;
generating, by at least one processor, a three-dimensional (3D) model of the patient's anatomy from the volume data;
placing a 3D model of an implant at a planned position or orientation at least partially within the 3D model of the patient's anatomy to create a 3D post-implantation model of the patient's anatomy;
following the placing, translating, by the one or more processors, the 3D post-implantation model of the patient's anatomy into a plurality of voxels of planned post-implantation volume data that correspond to the portion of the model of the implant positioned within the planned position or orientation, the translation including changing the assigned values of the plurality of voxels in the volume data to one or more new values in the planned post-implantation volume data;
creating, by the one or more processors, one or more predicted synthetic digital radiographs (DRs) from the planned post-implantation volume data; and
presenting the one or more predicted synthetic DRs on a display,
wherein the one or more predicted synthetic DRs show an image of the implant at the planned positions and/or orientations within the patient's anatomy.

2. The computer-implemented method of claim 1, further comprising generating the three-dimensional (3D) model of the patient's anatomy from image data generated from the volume data.

3. The computer-implemented method of claim 1, wherein a physical version of the implant is made from a material and the one or more new values are associated with a radiographic density of the material from which the physical version of the implant is made.

4. The computer-implemented method of claim 1, wherein a physical version of the implant is made from a material and the one or more new values is different from a value associated with a radiographic density of the material from which the physical version of the implant is made.

5. The computer-implemented method of claim 1, wherein the one or more predicted synthetic DRs is presented in a manner facilitating comparison with one or more intra-operative x-ray images taken of the patient.

6. The computer-implemented method of claim 5, further comprising presenting the one or more predicted synthetic DRs on a mixed reality device.

7. The computer-implemented method of claim 5, further comprising overlaying the one or more predicted synthetic DRs on the one or more intra-operative x-ray images.

8. The computer-implemented method of claim 1 wherein creating the one or more predicted synthetic DRs includes:
specifying a location of a virtual x-ray source relative to the planned post-implantation volume data;
specifying a location of a virtual x-ray detector relative to the planned post-implantation volume data; and
specifying one or more angles of virtual x-ray beams relative to the planned post-implantation volume data.

9. The computer-implemented method of claim 1, further comprising:
placing a model of a surgical tool at a planned position or orientation relative to the 3D model of the patient's anatomy to create the 3D post-implantation model of the patient's anatomy; and
wherein the plurality of voxels of translated planned post-implantation volume data correspond to the portion of the model of the implant positioned within the planned position or orientation and the model of the surgical tool positioned at the planned position.

10. The computer-implemented method of claim 9, wherein the one or more specified values are associated with a radiographic density of a material from which a physical version of the surgical tool is made.

11. The computer-implemented method of claim 1, wherein the pre-operative volume data comprises computed tomography (CT) data.

12. The computer-implemented method of claim 1, wherein the pre-operative volume data comprises predicted computed tomography (CT) data.

13. The computer-implemented method of claim 12, wherein the predicted CT data is derived from stereoradiographic imaging.

14. The computer-implemented method of claim 13, wherein the stereoradiographic imaging comprises EOS® biplanar X-ray imaging.

15. The computer-implemented method of claim 1, further comprising repositioning the 3D model of the patient's anatomy relative to one or more references.

16. The computer-implemented method of claim 15, wherein the patient's anatomy comprises a femur and the one or more references comprise a pelvis.

17. The computer-implemented method of claim 16, wherein the 3D model of the femur is repositioned such that an axis between a planned prosthetic femoral head center to a knee center is perpendicular to a medial lateral axis of the pelvis.

18. The computer-implemented method of claim 16, wherein the 3D model of the femur is repositioned such that an axis defined by a planned prosthetic femoral head center to a knee center according to the pelvic tilt (supine from the plan or a number entered from measurement of a standing view.

19. The computer-implemented method of claim 15, wherein the patient's anatomy comprises a femur and the one or more references comprise a planned acetabular component for a planned total hip arthroplasty (THA).
